(19) United States
(12) Patent Application Publication
(10) Pub. No.: US 2004/0029163 A1
(43) Pub. Date: Feb. 12, 2004

(54) Novel tissue-specific secretory polypeptide and dna thereof

(76) Inventors: Yasuaki Ito, Ibaraki (JP); Shinichi Mogi, Ibaraki (JP)

Correspondence Address: TAKEDA PHARMACEUTICALS NORTH AMERICA, INC, INTELLECTUAL PROPERTY DEPARTMENT, 475 HALF DAY ROAD, SUITE 500, LINCOLNSHIRE, IL, 60069, US

(21) Appl. No.: 10/450,875

(22) PCT Filed: Dec. 18, 2001
(86) PCT No.: PCT/JP01/11080

(30) Foreign Application Priority Data
Dec. 19, 2000 (JP) 2000-385192

(51) Int. Cl. C12Q 1/68; A01K 67/00; C07H 21/04; C12P 21/02; C12N 5/06; C07K 14/47

(52) U.S. Cl. 435/6; 435/69.1; 435/320.1; 435/325; 530/350; 536/23.5; 800/8

(57) ABSTRACT

The invention relates to a novel secretory protein. Specifically, the invention provides a novel secretory protein, a DNA encoding the same, a method for producing the protein, a medicine comprising the protein, an antibody against the protein, and the like.

The polypeptide of the invention and DNA encoding the same can be used for diagnosis, therapy and prevention of diseases such as respiratory diseases, cancers, immune diseases, infectious diseases, digestive tract diseases, circulatory diseases, endocrinopathy and bone and joint diseases.

### TECHNICAL FIELD

The present invention relates to a novel secretory polypeptide for regulating biogenic functions and DNA thereof.

### BACKGROUND ART

Cells secrete various kinds of proteins by their intrinsic mechanism irrespective of prokaryotic cells and eukaryotic cells. Particularly, while multicellular organisms (living organisms) exchange various information among the cells for maintaining differentiation, proliferation and homeostasis, many of various humoral factors that play a central role therein are secretory proteins and matured form thereof, and they are classified into hormones, neurotransmitters, cytokines and proliferation factors depending on their structural and functional features. Elucidation of genes encoding these secretory proteins and structures of the proteins are steadily advancing due to recent progress of recombinant DNA technologies and cell culture technologies. On the other hand, discoveries of such factors permit analysis of receptors expressed on the surface of the cells to be rapidly progressed while evoking elucidation of signal transduction mechanism in each cell that characterizes physiological functions of the cell. In many human diseases or pathology of model animals for various diseases, some abnormal expression of the humoral factors that should be essentially maintained homeostasis cause the disease, or may leads to exacerbation of the disease in many cases. Furthermore, the factors may serve as markers such as tumor markers that are observed to be specifically overexpressed in cancers, which may be applicable in diagnosis of various diseases. Accordingly, the expression control mechanism of the factor is an important target for drug development.

Analyses of total DNA, or genome, in living organisms have been completed with respect to about 70 biological species including archaebacteria, eubacteria, yeast, nematode, insects and plants, and analysis of human genes is almost close to its end (Nature 409, pp745-964, 2001; Science 291, pp1145-1434, 2001). Although the number of genes that had been estimated to be about 100,000 in several years ago was actually as small as about 40,000, it can be hardly concluded that all the genes encoding isolated secretory proteins or peptides have been elucidated considering the total number of genomes that have been analyzed. While information exchange among the cells should be able to be totally elucidated for comprehending life phenomena in an individual level, there are yet another possibilities that unknown humoral functional molecules other than known genes play important physiological roles, and discoveries of such substances have been urgently desired.

The invention aims to provide a novel secretory polypeptide for regulating biological functions (hereinafter, referred to as DRL138 polypeptide or simply as DRL138, or simply the polypeptide of the invention), an amide thereof, an ester thereof or a salt thereof (hereinafter, simply referred to as the partial peptide of the invention); DNA encoding the polypeptide or partial peptide (simply referred as DNA of the invention); a recombinant vector and a transformant; and a method for producing the polypeptide or partial peptide; pharmaceuticals containing the polypeptide or partial peptide or the DNA; and an antibody against the polypeptide or partial peptide.

### DISCLOSURE OF INVENTION

Isolation of novel secretory proteins for regulating biological functions affords new knowledge about the mechanisms of differentiation and proliferation of cells, biological protection and onset of cancers while further progressing elucidation of biological phenomena such as biogenesis and maintenance of homeostasis. Developments of novel medicines useful for prevention, diagnosis and therapy of various diseases may be expected by exhibiting an inhibitory activity or a promoting activity against the secretory proteins.

The inventors have succeeded, as a result of committed study for the first time, in cloning cDNA having a novel sequence from a human ovary cDNA library. The inventors also found that the protein encoded by the cDNA obtained is a secretory humoral factor produced in organs important in regulation of biological functions mainly in the trachea and lung of the fetus. The inventors have completed the invention through repeated studies based on this discovery.

The invention provides:

(1) A polypeptide, or an amide, an ester or a salt thereof comprising the same or substantially the same amino acid sequence as the amino acid sequence represented by SEQ ID NO: 4 (human full length sequence with no signals);

(2) The polypeptide, or an amide, an ester or a salt thereof according to (1), comprising an amino acid sequence represented by SEQ ID NO: 4 (human full length sequence with no signals), SEQ ID NO: 2 (human full length sequence containing signals), SEQ ID NO: 16 (human full length sequence+FLAG sequence), SEQ ID NO: 18 (mouse full length sequence containing signals), or SEQ ID NO: 20 (mouse full length sequence with no signals);

(3) A polypeptide, or an amide, an ester or a salt thereof comprising the same or substantially the same amino acid sequence as the amino acid sequence represented by SEQ ID NO: 6 (human mature sequence 1), SEQ ID NO: 8 (human mature sequence 2), SEQ ID NO: 22 (mouse mature sequence 1), or SEQ ID NO: 24 (mouse mature sequence 2);

(4) The polypeptide, or an amide, an ester or a salt thereof according to (3), comprising an amino acid sequence represented by SEQ ID NO: 6, SEQ ID NO: 8, SEQ ID NO: 22 or SEQ ID NO: 24;

(5) The polypeptide according to (1), or a partial peptide, an amide, an ester or a salt of the peptide according to (3);

(6) A polynucleotide comprising a polynucleotide encoding the polypeptide according to (1);

(7) The polynucleotide according to (6), which is a DNA;

(8) The DNA according to (7), having a base sequence represented by SEQ ID NO: 1, SEQ ID NO: 3, SEQ ID NO: 5, SEQ ID NO: 17, SEQ ID NO: 19 or SEQ ID NO: 21;

(9) A polynucleotide comprising a polynucleotide encoding the peptide according to (3);

(10) The polynucleotide according to (9), which is a DNA;

(11) The DNA according to (10), having a base sequence represented by SEQ ID NO: 7, SEQ ID NO: 9, SEQ ID NO: 23 or SEQ ID NO: 25;

(12) A recombinant vector comprising the polynucleotide according to (6) or (9);

(13) A transformant transformed by the recombinant vector according to (12);

(14) A method for producing the polypeptide, or the amide, ester or salt thereof according to (1), or the polypeptide, or the amide, ester or salt thereof according to (3), comprising the step of cultivating the transformant according to (13) to produce and accumulate the polypeptide according to (1) or the peptide according to (3);

(15) An antibody against the polypeptide, or the amide, ester or salt thereof according to (1), the peptide, or the amide, ester or salt thereof according to (3), or the partial peptide, or the amide, ester or salt thereof according to (5);

(16) A screening method of a compound or a salt thereof for promoting or inhibiting the activity of the polypeptide, or the amide, ester or salt thereof according to (1), the peptide, or the amide, ester or salt thereof according to (3), or the partial peptide, or the amide, ester or salt thereof according to (5), which comprises using the polypeptide, or the amide, ester or salt thereof according to (1), the peptide, or the amide, ester or salt thereof according to (3), or the partial peptide, or the amide, ester or salt thereof according to (5);

(17) A screening kit of the compound or the salt thereof for promoting or inhibiting the activity of the polypeptide, or the amide, ester or salt thereof according to (1), the peptide, or the amide, ester or salt thereof according to (3), or the partial peptide, or the amide, ester or salt thereof according to (5) comprising the polypeptide, or the amide, ester or salt thereof according to (1), the peptide, or the amide, ester or salt thereof according to (3), or the partial peptide, or the amide, ester or salt thereof according to (5);

(18) The compound or the salt thereof for promoting or inhibiting the activity of the polypeptide, or the amide, ester or salt thereof according to (1), the peptide, or the amide, ester or salt thereof according to (3), or the partial peptide, or the amide, ester or salt thereof according to (5), which is obtainable using the screening method according to (16) or the screening kit according to (17);

(19) A medicine comprising the compound or the salt thereof for promoting or inhibiting the activity of the amide, ester or salt thereof according to (1), the peptide, or the amide, ester or salt thereof according to (3), or the partial peptide, or the amide, ester or salt thereof according to (5), which is obtainable using the screening method according to (16) or screening kit according to (17);

(20) A preventive or therapeutic agent of respiratory diseases, cancers, immune diseases, infectious diseases, digestive tract diseases, circulatory diseases, bone and joint diseases or endocrinopathy comprising the compound or the salt thereof for promoting the activity of the amide, ester or salt thereof according to (1), the peptide, or the amide, ester or salt thereof according to (3), or the partial peptide, or the amide, ester or salt thereof according to (5), which is obtainable using the screening method according to (16), or the screening kit according to (17);

(21) A medicine comprising the polypeptide, or the amide, ester or salt thereof according to (1), or the peptide, or the amide, ester or salt thereof according to (3);

(22) A preventive or therapeutic agent of respiratory diseases, cancers, immune diseases, infectious diseases, digestive tract diseases, circulatory diseases, bone and joint diseases or endocrinopathy comprising the polypeptide, or the amide, ester or salt thereof according to (1), or the peptide, or the amide, ester or salt thereof according to (3);

(23) A medicine comprising the polynucleotide according to (6) or (9);

(24) The medicine according to (23), which is the preventive or therapeutic agent of respiratory diseases, cancers, immune diseases, infectious diseases, digestive tract diseases, circulatory diseases, bone and joint diseases or endocrinopathy;

(25) A diagnostic agent comprising the antibody according to (15);

(26) A medicine comprising the antibody according to (15);

(27) An antisense DNA having a base sequence or a part thereof complementary or substantially complementary to the DNA encoding the polypeptide according to (1) or the peptide according to (3), and having a function for suppressing the expression of the DNA;

(28) A medicine comprising the antisense DNA according to (27);

(29) A gene diagnosis agent comprising the polynucleotide according to (6) and (9), or the antisense DNA according to (27);

(30) A preventive or therapeutic method of respiratory diseases, cancers, immune diseases, infectious diseases, digestive tract diseases, circulatory diseases, bone and joint diseases or endocrinopathy comprising administering to mammals, an effective dose of the polypeptide, or the amide, ester or salt thereof according to (1), or the peptide, or the amide, ester or salt thereof according to (3);

(31) The preventive or therapeutic method of respiratory diseases, cancers, immune diseases, infectious diseases, digestive tract diseases, circulatory diseases, bone and joint diseases or endocrinopathy comprising administering an effective dose of the polynucleotide according to (6) or (9) to mammals.

(32) The preventive or therapeutic method of respiratory diseases, cancers, immune diseases, infectious diseases, digestive tract diseases, circulatory diseases, bone and joint diseases or endocrinopathy comprising administering, to mammals, an effective dose of the compound or the salt thereof for promoting or inhibiting the activity of the polypeptide, or the amide, ester or salt thereof according to (1), the peptide, or the amide, ester or salt thereof according to (3), or the partial peptide, or the amide, ester or salt thereof according to (5), which is obtainable using the screening method according to (16) or the screening kit according to (17);

(33) Use of the polypeptide, or the amide, ester or salt thereof according to (1), or the peptide, or the amide, ester or salt thereof according to (3) for producing the preventive or therapeutic agent of respiratory diseases, cancers, immune diseases, infectious diseases, digestive tract diseases, circulatory diseases, bone and joint diseases or endocrinopathy;

(34) Use of the polynucleotide according to (6) or (9) for producing the preventive or therapeutic agent of respiratory diseases, cancers, immune diseases, infectious diseases, digestive tract diseases, circulatory diseases, bone and joint diseases or endocrinopathy;

(35) Use of the compound or the salt thereof for promoting the activity of the polypeptide, or the amide, ester or salt thereof according to (1), the peptide, or the amide, ester or salt thereof according to (3), or the partial peptide, or the amide, ester or salt thereof according to (5), which is obtainable using the screening method according to (16) or screening kit according to (17) for producing the preventive or therapeutic agent of respiratory diseases, cancers, immune diseases, infectious diseases, digestive tract diseases, circulatory diseases, bone and joint diseases or endocrinopathy;

(36) A transgenic non-human mammal having the exogenous DNA according to (7) or (10) or mutated DNA thereof;

(37) The non-human mammal according to (36), wherein the non-human mammal is a rodent;

(38) The non-human mammal according to (37), wherein the rodent is a mouse;

(39) A recombinant vector comprising the exogenous DNA or the mutated DNA thereof according to (7) or (10), which is capable of being expressed in the non-human mammal;

(40) An embryonic stem cell of the non-human mammal, in which the DNA according to (7) or (10) is inactivated;

(41) The embryonic stem cell according to (40), wherein the DNA is inactivated by introducing a reporter gene;

(42) The embryonic stem cell according to (40), which is resistant to neomycin;

(43) The embryonic stem cell according to (40), wherein the non-human mammal is a rodent;

(44) The embryonic stem cell according to (43), wherein the rodent is a mouse;

(45) A non-human mammal deficient in DNA expression, in which the DNA according to (7) or (10) is inactivated;

(46) The non-human mammal according to (45), wherein the DNA is inactivated by introducing the reporter gene, which is able to be expressed under the control of a promoter to the DNA;

(47) The non-human mammal according to (46), wherein the mammal is a rodent;

(48) The non-human mammal according to (47), wherein the rodent is a mouse;

(49) A method for screening a compound or a salt thereof for promoting or inhibiting the activity of the promoter to the DNA according to (7) or (10), which comprises administering test compound to the non-human mammal according to claim 46 and detecting the expression of the reporter gene.

The invention further provides:

(50) The polypeptide, or the amide, ester or salt thereof according to (1), wherein the amino acid sequence substantially the same as the amino acid sequence represented by SEQ ID NO: 4 is an amino acid sequence having at least about 50% (preferably at least about 60%, further preferably at least about 70%, more preferably at least about 80%, in particular preferably at least about 90%, and most preferably at least about 95%) of homology with the amino acid sequence of SEQ ID NO: 4; and

(51) The polypeptide, or the amide, ester or salt thereof according to (1), wherein the amino acid sequence substantially the same as the amino acid sequence represented by SEQ ID NO: 4 is [1] an amino acid sequence in which one or at least two (preferably about 1 to 30, further preferably about 1 to 20, more preferably 1 to 10, particularly 1 to 5, and most preferably 1 or 2) amino acids are deleted in the amino acid sequence represented by the SEQ ID NO: 4, [2] an amino acid sequence in which one or at least two (preferably about 1 to 30, further preferably about 1 to 20, more preferably 1 to 10, particularly 1 to 5, and most preferably 1 or 2) amino acids are added in the amino acid sequence represented by the SEQ ID NO: 4, or [3] an amino acid sequence in which one or at least two (preferably about 1 to 30, further preferably about 1 to 20, more preferably 1 to 10, particularly 1 to 5, and most preferably 1 or 2) amino acids are substituted with other amino acids in the amino acid sequence represented by the SEQ ID NO: 4, and [4] an amino acid sequence as a combination thereof.

The polypeptide, partial peptide and DNA of the invention may be used as a molecular weight marker and tissue marker, and applicable to chromosome mapping, identification of hereditary diseases, diagnosis of pathology, and basic researches such as design of primers and probes.

### BRIEF DESCRIPTION OF THE DRAWINGS

FIG. 1 shows the results of expression of DRL138h-FLAG in COS-7 cells, and the pattern of the expression products.

Sample including each lane is as follows: 1: concentrated culture medium of COS-7 (DRL138h-FLAG); 2: COS-7 (DRL138h-FLAG) cell; 3: concentrated culture medium of COS-7 (control); 4: COS-7 (control) cell; 5: culture medium of COS-7 (DRL138h-FLAG); 6: culture medium of COS-7 (control)

FIG. 2 is a comparison of the amino acid sequences between human DRL138 (hDRL138) and mouse DRL138 (mDRL138).

### BEST MODE FOR CARRYING OUT THE INVENTION

The polypeptide comprising the same or substantially the same amino acid sequence as the amino acid sequence represented by SEQ ID NO: 4 (referred to as the polypeptide of the invention hereinafter; the polypeptide comprising the same or substantially the same amino acid sequence as the amino acid sequence represented by SEQ ID NO: 4, or the amide, ester or salt thereof may be collectively referred to as the polypeptide of the invention) may be a polypeptide derived from cells (hepatic cell, splenic cell, nerve cell, glia cell, pancreas β-cell, bone marrow cell, mesangial cell, Langerhans cell, epidermal cell, epithelial cell, endothelial cell, fibroblast, fibrous cell, muscle cell, fat cell, immune cells (for example macrophage, T-cell, B-cell, natural killer cells mast cell, neutrophil, basophil, eosinophil and monocyte), megakaryocyte, synovial cell, chondrocyte, osteocyte, osteoblast, osteoclast, mammary gland cell and interstitial cell, or precursor cells, stem cells or cancer cells of these cells); or all the tissues containing these cells, for example, brain, each site of brain (for example, olfactory bulb, amygdaloid nucleus, basal ganglia, hippocampus, thalamus, hypothalamus, cerebral cortex, medulla oblongata and cerebellum), spinal cord, pituitary gland, stomach, pancreas, kidney, liver, genital gland, thyroid gland, gall bladder, bone marrow, adrenal gland, skin, muscle, lung, digestive tract (for example, large intestine and small intestine), blood vessel, heart, thymus, spleen, salivary gland, peripheral blood, prostate gland, testis, ovary, placenta, uterus, bone, cartilage, joint and skeletal muscle of human and other warm-blooded animals (for example, guinea pig, rat, mouse, avian, rabbit, swine, sheep, bovine and monkey). The polypeptides may also be a recombinant polypeptide, or a synthetic polypeptide.

When the polypeptide of the invention has a signal peptide (specifically, a polypeptide comprising the same or substantially the same amino acid sequence as the amino acid sequence having a signal sequence consisting of 15 to 30 amino acid residues at the N-terminal of SEQ ID NO: 4 or SEQ ID NO: 20 such as SEQ ID NO: 2 or SEQ ID NO: 18), the polypeptide can be efficiently secreted out of the cell.

The term “substantially the same” means that physiological characteristics such as preventive and therapeutic activity (action) for the respiratory diseases, alimentary diseases, cancer, immune diseases, infectious diseases, digestive tract diseases, endocrinopathy, and bone and joint diseases are substantially the same. While substitution, deletion, addition or insertion of the amino acids often do not bring about large changes in the physiological characteristics and chemical properties of the polypeptide, such polypeptides subjected to substitution, deletion, addition or insertion might be considered to be substantially the same as the polypeptide before modification. The substantially the same substituent of the amino acid in the amino acid sequence in concern may be selected from other amino acids in the class to which the amino acid belongs.

Examples of non-polar (hydrophobic) amino acids include alanine, leucine, isoleucine, valine, proline, phenylalanine, tryptophane and methionine. Examples of polar (neutral) amino acids include glycine, serine, threonine, cysteine, tyrosine, asparagine and glutamine. Examples of positively charged (basic) amino acids include arginine, lysine and histidine. Examples of negatively charged (acidic) amino acids include aspartic acid and glutamic acid.

While the amino acid sequences substantially the same as the amino acid sequence represented by SEQ ID NO: 4 are not particularly restricted so long as the polypeptide comprising the amino acid sequence has substantially the same activity (property) as the polypeptide comprising the amino acid sequence represented by SEQ ID NO: 4, the amino acid sequence has, for example, at least about 50%, preferably at least about 60%, further preferably at least about 70%, more preferably at least about 80%, particularly at least about 90%, and most preferably at least about 95% of homology with the amino acid sequence represented by SEQ ID NO: 4. More specifically, the amino acid sequence is represented by SEQ ID NO: 16, SEQ ID NO: 18 or SEQ ID NO: 20.

Examples of the substantially equivalent activity (property) include an action as a secreted humoral factor. The term “substantially equivalent” means that the property is qualitatively identical. Accordingly, while it is preferable that properties such as secretion action and solubility are equivalent (about 0.1 to 100 times, preferably about 0.5 to 10 times, and more preferably 0.5 to 2 times), the extent of these properties and quantitative factors such as the molecular weight of the polypeptide may be different.

The substantially equivalent activity (property) means that, for example, preventive and therapeutic actions for respiratory diseases, cancers, immune diseases, infectious diseases, digestive tract diseases, circulatory diseases, endocrinopathy, and bone and joint diseases are qualitatively identical.

Examples of the polypeptide comprising substantially the same amino acid sequence as the amino acid sequence represented by SEQ ID NO: 4 include (a) an amino acid sequence in which one or at least two (preferably 1 to 30, further preferably 1 to 20, more preferably 1 to 10, particularly 1 to 5, and most preferably 1 or 2) amino acids are deleted in the amino acid sequence represented by the SEQ ID NO: 4 or SEQ ID NO: 20, (b) an amino acid sequence in which one or at least two (preferably 1 to 30, further preferably 1 to 20, more preferably 1 to 10, particularly 1 to 5, and most preferably 1 or 2) amino acids are added in the amino acid sequence represented by the SEQ ID NO: 4 or SEQ ID NO: 20, or (c) an amino acid sequence in which one or at least two (preferably 1 to 30, further preferably about 1 to 20, more preferably 1 to 10, particularly 1 to 5, and most preferably 1 or 2) amino acids are substituted with other amino acids in the amino acid sequence represented by the SEQ ID NO: 4 or SEQ ID NO: 20, and (d) an amino acid sequence as a combination thereof or a so-called mutein.

While insertion, deletion or substitution site is not particularly restricted when the amino acid sequence is inserted, deleted or substituted as described above, examples thereof are the sites except basic amino acid pairs in SEQ ID NO: 4 or SEQ ID NO: 20.

While any peptides may be available as the partial peptide of the polypeptide of the invention (partial peptide of the invention) so long as it is any one of the partial peptides of the polypeptide of the invention, examples of the preferably used partial peptide include those having substantially equivalent activity to the polypeptide of the invention (the term “substantially equivalent activity” has the same meaning as described above).

However, unlike the polypeptide of the invention, since the partial peptide of the invention can be used as an antigen for preparing an antibody, it is not necessarily to have the activity involved in the polypeptide of the invention.

Examples of the partial peptide of the invention include (a) the partial peptide of the polypeptide of the invention comprising the same or substantially the same amino acid sequence as the amino acid sequence represented by SEQ ID NO: 6 or SEQ ID NO: 22 (more specifically, the partial peptide of the polypeptide of the invention comprising the same or substantially the same amino acid sequence as the amino acid sequence represented by SEQ ID NO: 6 or SEQ ID NO: 22; particularly the peptide comprising the amino acid sequence represented by SEQ ID NO: 6 or SEQ ID NO: 22), and (b) the partial peptide of the polypeptide of the invention comprising the same or substantially the same amino acid sequence as the amino acid sequence represented by SEQ ID NO: 8 or SEQ ID NO: 24 (more specifically, the partial peptide of the polypeptide of the invention comprising the same or substantially the same amino acid sequence as the amino acid sequence represented by SEQ ID NO: 8 or SEQ ID NO: 24; particularly the peptide comprising the amino acid sequence represented by SEQ ID NO: 8 or SEQ ID NO: 24).

The term “substantially the same” as used herein has the same meaning as “substantially the same” used in the description of the polypeptide of the invention.

While the amino acid sequence substantially the same as the amino acid sequence represented by SEQ ID NO: 6, the amino acid sequence substantially the same as the amino acid sequence represented by SEQ ID NO: 8, the amino acid sequence substantially the same as the amino acid sequence represented by SEQ ID NO: 22, and the amino acid sequence substantially the same as the amino acid sequence represented by SEQ ID NO: 24 are not particularly restricted so long as the polypeptide comprising the amino acid sequence has substantially the same activity (property) as the partial peptide comprising the amino acid sequence represented by SEQ ID NO: 6, the partial peptide comprising the amino acid sequence represented by SEQ ID NO: 8, and the partial peptide comprising the amino acid sequence represented by SEQ ID NO: 22, examples of the amino acid sequence have at least about 70%, preferably at least about 80%, more preferably at least about 90%, further preferably at least about 95%, and most preferably at least about 98% of homology with the amino acid sequence represented by SEQ ID NO: 6, the amino acid sequence represented by SEQ ID NO: 8, the amino acid sequence represented by SEQ ID NO: 22, and the amino acid sequence represented by SEQ ID NO: 24.

Examples of the partial peptide comprising the same or substantially the same amino acid sequence as the amino acid sequence represented by SEQ ID NO: 6, SEQ ID NO: 8, SEQ ID NO: 22, or SEQ ID NO: 24 include (1) [1] the amino acid sequence in which one or at least two amino acids (for example 1 to 10, preferably 1 to 5, and more preferably one or at least two amino acids) of the amino acid sequences represented by SEQ ID NO: 6 are deleted, [2] the amino acid sequence in which one or at least two amino acids (for example 1 to 10, preferably 1 to 5, and more preferably one or at least two amino acids) of the amino acid sequences represented by SEQ ID NO: 6 are added, [3] the amino acid sequence in which one or at least two amino acids (for example 1 to 10, preferably 1 to 5, and more preferably one or at least two amino acids) of the amino acid sequences represented by SEQ ID NO: 6 are substituted with other amino acids, and [4] the partial peptide comprising the amino acid sequences as a combination thereof; (2) [1] the amino acid sequence in which one or at least two amino acids (for example 1 to 5, and preferably one or two amino acids) of the amino acid sequences represented by SEQ ID NO: 8 are deleted, [2] the amino acid sequence in which one or at least two amino acids (for example 1 to 5, and preferably one or two amino acids) of the amino acid sequences represented by SEQ ID NO: 8 are added, [3] the amino acid sequence in which one or at least two amino acids (for example 1 to 5, and preferably one or two amino acids) of the amino acid sequences represented by SEQ ID NO: 8 are substituted with other amino acids, and [4] the partial peptide comprising the amino acid sequences as a combination thereof; (3) [1] the amino acid sequence in which one or at least two amino acids (for example 1 to 5, and preferably one or two amino acids) of the amino acid sequences represented by SEQ ID NO: 22 are deleted, [2] the amino acid sequence in which one or at least two amino acids (for example 1 to 5, and preferably one or two amino acids) of the amino acid sequences represented by SEQ ID NO: 22 are added, [3] the amino acid sequence in which one or at least two amino acids (for example 1 to 5, and preferably one or two amino acids) of the amino acid sequences represented by SEQ ID NO: 22 are substituted with other amino acids, and [4] the partial peptide comprising the amino acid sequences as a combination thereof; and (4) [1] the amino acid sequence in which one or at least two amino acids (for example 1 to 5, and preferably one or two amino acids) of the amino acid sequences represented by SEQ ID NO: 24 are deleted, [2] the amino acid sequence in which one or at least two amino acids (for example 1 to 5, and preferably one or two amino acids) of the amino acid sequences represented by SEQ ID NO: 24 are added, [3] the amino acid sequence in which one or at least two amino acids (for example 1 to 5, and preferably one or two amino acids) of the amino acid sequences represented by SEQ ID NO: 24 are substituted with other amino acids, and [4] the partial peptide comprising the amino acid sequences as a combination thereof, or a so-called mutein.

While the insertion, deletion or substitution site is not particularly restricted when the amino acid sequence is inserted, deleted or substituted, examples thereof are the sites except basic amino acids in the sequence.

The polypeptide of the invention, and the partial peptide of the invention include those in which substituents on the side chains of the amino acids in the molecule are protected with appropriate protective groups, or so-called complex peptides such as glyco-polypeptides in which sugar chains are bound to the polypeptide.

The polypeptide of the invention, or the partial peptide of the invention includes those having any exogenous peptide sequences (for example FLAG, His tag, HA tag and HSV tag) that may serve as epitope at the N- or C-terminals.

Examples of such (poly)peptide include the polypeptide comprising the amino acid sequence represented by SEQ ID NO: 16.

The polypeptide of the invention, and the partial peptide of the invention are defined to have a N-terminal (amino terminal) at the left end and a C-terminal (carboxyl terminal) at the right end according to the traditional notation of the peptide. While the polypeptide of the invention including the polypeptide comprising the amino acid sequence represented by SEQ ID NO: 4 usually has the carboxyl group (—COOH) or carboxylate (—COO⁻) at the C-terminal, it may have an amide (—CONH₂) or ester (—COOR).

Examples of R in the ester include C₁₋₆ alkyl groups such as methyl, ethyl, n-propyl, isopropyl or n-butyl group, C₃₋₈ cycloalkyl groups such as cyclopentyl or cyclohexyl group, C₆₋₁₂ aryl groups such as phenyl or α-naphthyl group, phenyl C₁₋₂ alkyl group such as benzyl or phenethyl group, α-naphthyl-C₁₋₂ alkyl groups [such as α-naphthylmethyl group], or C₇₋₁₄ aralkyl groups, as well as pyvaloyloxumethyl group frequently used in orally administered esters.

When the polypeptide of the invention, and the partial peptide of the invention have the carboxyl group (or carboxylate) at the site other than the C-terminal, those having amidated or esterified carboxyl groups are also included in the polypeptide of the invention and partial peptide of the invention. The esters used in this case include C-terminal esters described above.

Preferably used partial peptides of the invention include amides (for example the C-terminal carboxyl group (—COOH) is amidated (—CONH₂)) of the partial peptide comprising substantially the same amino acid sequence as the amino acid sequence represented by SEQ ID NO: 8 or SEQ ID NO: 24.

Furthermore, the polypeptide of the invention, and the partial peptide of the invention include those in which amino groups of the N-terminal amino acid residues (for example methionine residue) are protected with protective groups (for example, C₁₋₆ acyl group including C₁₋₆ alkanoyl group such as formyl group and acetyl group); those in which the glutamine residue formed by being cleaved at the N-terminal is converted into pyrroglutamine in the body; those in which substituents (for example —OH, —SH, amino group, imidazole group, indole group and guanidino group) on the side chain of the amino acid in the molecule are protected with an appropriate protective group (for example C₁₋₆ acyl group including C₁₋₆ alkanoyl group such as formyl group and acetyl group); or complex polypeptides such as so-called glycopolypeptides in which sugar chains are bonded to the polypeptide.

Salts with physiologically acceptable acids (inorganic salts and organic salts) and bases (alkali metal salts) are used as the salt of the polypeptide of the invention, and physiologically acceptable acid addition salts are preferable among them. Examples of such salts include salts of inorganic acids (hydrochloric acid, phosphoric acid, hydrobromic acid and sulfuric acid), and salts of organic acids (acetic acid, formic acid, propionic acid, fumaric acid, maleic acid, succinic acid, tartaric acid, citric acid, malic acid, oxalic acid, benzoic acid, methanesulfonic acid and benzene sulfonic acid).

The polypeptide of the invention, and the partial peptide of the invention can be produced by publicly known purification methods of polypeptides (proteins) from cells or tissues of human or other warm-blooded animals, or by cultivating transformant containing DNA encoding the polypeptide of the invention and partial peptide of the invention. The polypeptide of the invention, and the partial peptide of the invention can be also produced by peptide synthesis methods as will be described below.

When the polypeptide of the invention, and the partial peptide of the invention are produced from the cells or tissues of human and other mammals, the cells or tissues of human and other mammals are extracted with an acid or the like after homogenization, and the extract of the polypeptide of the invention or the partial peptide of the invention is purified and isolated by a combination of chromatography such as reversed phase chromatography and ion exchange chromatography.

Usually, a commercially available resin for polypeptide (protein) synthesis is used for synthesis of the polypeptide of the invention or the partial peptide of the invention, or the salts or amides thereof. Examples of such resin include chloromethyl resin, hydroxymethyl resin, benzhydryl-amine resin, aminomethyl resin, 4-benzyloxy benzylalcohol resin, 4-methylbenzhydryl amine resin, PAM resin, 4-hydroxymethyl methylphenylacetamide methyl resin, polyacrylamide resin. 4-(2′,4′-dimethoxyphenyl-hydroxymethyl) phenoxy resin, and 4-(2′,4′-dimethoxyphenyl-Fmoc-aminoethyl)phenoxy resin. Amino acids in which α-amino groups and side chain functional groups are appropriately protected are allowed to condense on the resin as described above following the sequence of the desired polypeptide according to various publicly known methods. The protective groups are removed simultaneously with cleaving the polypeptide from the resin at the end of the reaction. Subsequently, intramolecular disulfide bonds are formed in a highly diluted solution to obtain the desired polypeptide or an amide thereof.

While various activating reagents available for peptide synthesis may be used for condensation of the protected amino acids, carbodiimide is particularly preferable. Examples of carbodiimide available include DCC, N,N′-diisopropylcarbodiimide, and N-ethyl-N′-(3-dimethylaminopropyl)carbodiimide. The amino acids may be activated by directly adding protected amino acids to the resin together with a racemization preventive agent (for example HOBt and HOOBt), or by may be added to the resin after previously activating the protected amino acids as corresponding acid anhydrides, HOBt esters or HOOBt esters.

Solvents to be used for activation of the protected amino acids and condensation with the resin may be appropriately selected from the solvents known to be available for the condensation reaction of the polypeptide (protein). Examples of them include amides such as N,N-dimethylformamide, N,N-dimethylacetamide and N-methylpyrrolidone, halogenated hydrocarbons such as methylene chloride and chloroform, alcohols such as trifluoroethanol, sulfoxides such as dimethylsulfoxide, pyridine, ethers such as dioxane and tetrahydrofuran, nitriles such as acetonitrile and propionitrile, esters such as methyl acetate and ethyl acetate, or an appropriate mixture thereof. The reaction temperature is appropriately selected from the temperatures known to be available for polypeptide (protein) bond forming reactions, and this temperature is usually selected in the range of about −20 to 50° C. A 1.5 to 4 fold excess of the activated amino acid derivatives is usually used. When condensation is judged to be sufficient from a test result using a ninhydrin reaction, sufficient condensation is possible by repeating the condensation reaction without eliminating the protective groups. When sufficient condensation is not achieved even after repeated reactions, unreacted amino acids are acetylated using acetic anhydride or acetyl imidazole so that the unreacted amino acids do not adversely affect the reaction thereafter.

Examples of the protective group of the amino groups of the starting material include Z, Boc, t-pentyloxycarbonyl, isobornyloxycarbonyl, 4-methoxybenzyloxyxarbonyl, Cl-Z, Br-Z, Br-Z, adamantyl oxyxarbonyl, trifluoroacetyl, phthaloyl, formyl, 2-nitrophenylsulfenyl, diphenylphosphinothioyl and Fmoc groups.

The carboxyl group can be protected, for example, by alkylesterification (for example esterification by linear, branched or cyclic alkylesters such as methyl, ethyl, propyl, butyl, t-butyl, cyclopentyl, cyclohexyl, cycloheptyl, cyclooctyl, 2-adamantyl), alalkyl esterification (for example esterification by benzyl ester, 4-nitrobenzyl ester, 4-methoxybenzyl ester, 4-chlorobenzyl ester or benzhydryl ester), phenacyl esterification, and conversion into hydrazide compounds by benzyloxycarbonyl, t-butoxycarbonyl and trityl groups.

The hydroxyl group of serine can be protected by esterification or etherification. Examples of the group suitable for esterification include lower alkanoyl (C₁₋₆) groups such as acetyl group, arolyl groups such as benzoyl group, and groups derived from carbonate group such as benzyloxycarbonyl group and ethoxycarbonyl group. Examples of the group suitable for etherification include benzyl group, tetrehydropyranyl group and t-butyl group.

Bzl, Cl₂-Bzl, 2-nitrobenzyl, Br-Z and t-butyl groups are used, for example, as the protective group of phenolic hydroxyl group of tyrosine.

Tos, 4-methoxy-2,3,6-trimethylbenzenesulfonyl, DNP, benzyloxymethyl, Boc, Trt and Fmoc groups are used, for example, as the protective groups of imidazole of histidine.

Examples of the activated carboxyl group of the starting material include corresponding acid anhydrides, azides and active esters (for example, esters with alcohols such as pentachlorophenol, 2,4,5-trichlorophenol, 2,4-dinitrophenol, cyanomethyl alcohol, paranitrophenol, HONB, N-hydrixysuccimide, N-hydroxyphthalimide and HOBt). Examples of the activated amide group of the starting material include corresponding phosphoric acid amide.

The protective group is removed (eliminated), for example, by heterogeneous reduction in a hydrogen stream in the presence of a catalyst such as Pd-black or Pd-carbon; acid treatment with anhydrous hydrogen fluoride, methanesulfonic acid, trifluoromethane sulfonic acid, trifluoroacetic acid or a mixture thereof; base treatment with diiopropylethyl amine, triethylamine, piperidine or piperazine; or reduction by sodium in liquid ammonia. While the elimination reaction in the acid treatment is usually carried out at a temperature of −20 to 40° C., addition of cation scavenger such as anisole, phenol, thioanisole, methacresol, paracresol, dimethylsulfoxide, 1,4-butanedithiol and 1,4-butanedithiol is effective. The 2,4-dinitrophenol group used for the imidazole protective group of histidine is removed by a thiophenol treatment, and the formyl group used for the indole protective group of tryptophane is removed by an acid treatment in the presence of 1,2-ethanediol or 1,4-butanediol as well as by an alkali treatment with a dilute sodium hydroxide solution or dilute aqueous ammonia.

Protection and protective groups of functional groups that should not be involved in the reaction of the starting material, elimination of the protective groups, and activation of the functional groups that are involved in the reaction may be appropriately selected from publicly known groups and publicly known means.

In another method for obtaining the polypeptide of the invention, or the partial peptide of the invention, the α-carboxylic group of the amino acid at the carboxyl terminal is protected at first by amidation followed by extending the peptide (polypeptide) chain to a desired chain length to the N-terminal of the peptide chain. Subsequently, the polypeptide from which only the protective group of the α-amino group at the N-terminal of the peptide chain is eliminated, and the polypeptide from which only the protective group of the carboxyl group at the C-terminal of the polypeptide is eliminated, are produced, and both polypeptides are allowed to condense in a mixed solvent as described above. Details of the condensation reaction are as described above. All the protective groups are removed after purification of the protected polypeptides obtained by condensation, thereby obtaining the desired crude polypeptide. The crude polypeptide is purified by arbitrarily using various purification methods known in the art, and an amide of the desired polypeptide can be obtained by lyophilizing the main fraction.

For obtaining esters of the polypeptide of the invention, and esters of the partial peptide of the invention, amino acid esters are formed by condensation of desired alcohols with the α-carboxylic group of the terminal amino acid, followed by the same process as obtaining the amides of the polypeptide of the invention, and esters of the partial peptide of the invention.

The polypeptide of the invention, and esters of the partial peptide of the invention can be produced by publicly known methods for peptide synthesis. Either a solid phase synthesis method or the liquid phase synthesis method may be employed as the peptide synthesis methods. A partial peptide constituting the partial peptide of the invention (a partial peptide capable of constituting the partial peptide of the invention) or an amino acid is condensed with a residual part of the peptide, and protective groups are eliminated when the product has the protective groups, thereby producing the desired peptide. The condensation methods and the elimination methods of the protective groups are described in the following references [1] to [5].

[1] Bodanszky and M. A. Ondetti, Peptide Synthesis, Interscience Publishers, New York (1966);

[2] Schroeder and Leubke, The Peptide, Academic Press, New York (1965);

[3] Nobuo Izumiya et. al., “Pepuchido Gosei no Kiso to Jikken (Basis and Experiment of Peptide Synthesis)”, Maruzen Co., (1975);

[4] Yoshiaki Yajima and Shunpei Sakakibara, “Seikagaku Jikken Koza (Textbook of Experimental Biochemistry), Vol. I: Tanpakusitsu no Kagaku (Chemistry of Protein) IV”, 1977; and

[5] Haruaki Yajima, ed., “Zoku Iyakuhin no Kaihatu (Development of Medicines), Suppl. Edition Vol 14; Pepuchido Gosei (Peptide Synthesis), Hirokawa Shoten.

The polypeptide of the invention, and the partial peptide of the invention can be purified and isolated by a combination of conventional purification methods, for example solvent extraction, distillation, column chromatography, liquid chromatography and recrystallization. When the polypeptide obtained by the methods above is a free form, it can be converted into an appropriate salt by a publicly known method or according to the method. On the contrary, when the peptide is obtained as a salt, it may be converted into a free form or another salt thereof by a publicly known method or according to the method.

Any DNA encoding the polypeptide of the invention, and the partial peptide of the invention (collectively referred to as DNA of the invention) may be available so long as the DNA contains base sequences encoding the polypeptide of the invention, and the partial peptide of the invention. Otherwise, genome DNA, cDNA derived from the cells and tissues described above, and synthetic DNA may be also used.

As vector used for libraries, any of a bacteriophage, plasmid, cosmid and phagemide may be available. The vector can be amplified directly by Reverse Transcriptase Polymerase Chain Reaction (referred to as RT-PCR hereinafter) using total RNA or mRNA prepared from the cells and tissues described above.

Any DNA encoding the polypeptide of the invention may be used so long as it contains the DNA encoding the polypeptide having the substantially equivalent activity (property) as the polypeptide of the invention (for example, immunogenicity; preventive or therapeutic activity (action) for respiratory diseases, cancers, immune diseases, infectious diseases, digestive tract diseases, circulatory diseases, endocrinopathy, and bone and joint diseases), and it is a DNA encoding polypeptide having substantially equivalent property as the polypeptide of the invention.

Examples of the DNA encoding the polypeptide of the invention include:

[1] DNA comprising the DNA having the base sequence represented by SEQ ID NO: 5;

[2] DNA comprising the DNA having the base sequence represented by SEQ ID NO: 3;

[3] DNA comprising the DNA having the base sequence represented by SEQ ID NO: 1;

[4] DNA comprising the DNA having the base sequence represented by SEQ ID NO: 17;

[5] DNA comprising the DNA having the base sequence represented by SEQ ID NO: 19; and

[6] DNA comprising the DNA having the base sequence represented by SEQ ID NO: 21.

Further, among them, “DNA comprising the DNA having the base sequence represented by SEQ ID NO: 3”, “DNA comprising the DNA having the base sequence represented by SEQ ID NO: 1”, and “DNA comprising the DNA having the base sequence represented by SEQ ID NO: 17” are included in the examples of “DNA comprising the DNA having the base sequence represented by SEQ ID NO: 5”, and “DNA comprising the DNA having the base sequence represented by SEQ ID NO: 19” is included in the examples of “DNA comprising the DNA having the base sequence represented by SEQ ID NO: 21”.

A DNA hybridizable with the base sequences represented by SEQ ID NO: 5, SEQ ID NO: 1, SEQ ID NO: 3, SEQ ID NO: 17, SEQ ID NO: 19, or SEQ ID NO: 21 under a highly stringent condition is also included in the DNA encoding the polypeptide of the invention. The DNA having at least about 70%, preferably at least about 80%, and more preferably at least about 90% of homology with the base sequences represented by SEQ ID NO: 5, SEQ ID NO: 1, SEQ ID NO: 3, SEQ ID NO: 17, SEQ ID NO: 19, or SEQ ID NO: 21 may be used in the invention.

Examples of the DNA hybridizable with the base sequences represented by SEQ ID NO: 5, SEQ ID NO: 1, SEQ ID NO: 3, SEQ ID NO: 17, SEQ ID NO: 19, or SEQ ID NO: 21 under highly stringent conditions include DNA hybridizable with the base sequences represented by SEQ ID NO: 5, SEQ ID NO: 1, SEQ ID NO: 3, SEQ ID NO: 17, SEQ ID NO: 19, or SEQ ID NO: 21 under highly stringent conditions, and encode polypeptide having the substantially equivalent property (the same meaning as described above) to the polypeptide of the invention.

Hybridization can be performed by the publicly known method, or according to the method, for example according to the method described in Molecular Cloning, 2nd edition (J. Sambrook et. al., Cold Spring Harbor Lab. Press, 1989). When a commercially available library is used, hybridization may be performed according to the method described in the attached instructions. More preferably, hybridization is performed under a highly stringent condition.

The highly stringent condition means that the sodium concentration is about 19 to 40 mM, preferably about 19 to 20 mM, and the temperature is about 50 to 70° C., preferably about 60 to 65° C.

The DNA having the base sequence represented by SEQ ID NO: 5 is used as the DNA encoding the polypeptide of the invention having the amino acid sequence represented by SEQ ID NO: 4; the DNA having the base sequence represented by SEQ ID NO: 3 is used as the DNA encoding the polypeptide of the invention having the amino acid sequence represented by SEQ ID NO: 2; the DNA having the base sequence represented by SEQ ID NO: 17 is used as the DNA encoding the polypeptide of the invention having the amino acid sequence represented by SEQ ID NO: 16; the DNA having the base sequence represented by SEQ ID NO: 19 is used as the DNA encoding the polypeptide of the invention having the amino acid sequence represented by SEQ ID NO: 18; and the DNA having the base sequence represented by SEQ ID NO: 21 is used as the DNA encoding the polypeptide of the invention having the amino acid sequence represented by SEQ ID NO: 20. The DNA having the base sequence represented by SEQ ID NO: 1 is the DNA comprising the DNA having the base sequence represented by SEQ ID NO: 3 as the DNA encoding the polypeptide of the invention having the amino acid sequence represented by SEQ ID NO: 2, as will be described below.

Any DNAs may be used as the DNA encoding the partial peptide of the invention, so long as they encode the partial peptide of the invention.

Examples of the DNA encoding the partial peptide of the invention include:

[1] DNAs comprising the DNA having the base sequence represented by SEQ ID NO: 7;

[2] DNAs comprising the DNA having the base sequence represented by SEQ ID NO: 9;

[3] DNAs comprising the DNA having the base sequence represented by SEQ ID NO: 23; and

[4] DNAs comprising the DNA having the base sequence represented by SEQ ID NO: 25.

DNAs hybridizable with the base sequences represented by SEQ ID NO: 7, SEQ ID NO: 9, SEQ ID NO: 23 or SEQ ID NO: 25 under a highly stringent condition may be included in the examples of the DNA encoding the partial peptide of the invention. DNAs comprising the base sequence having at least about 70%, preferably at least about 80%, and more preferably at least about 90% of homology with the base sequence represented by SEQ ID NO: 7, SEQ ID NO: 9, SEQ ID NO: 23 or SEQ ID NO: 25 may be used in the invention.

Hybridization can be performed by the method known in the art, or according to the method, for example according to the method described in Molecular Cloning, 2nd edition (J. Sambrook et. al., Cold Spring Harbor Lab. Press, 1989). When a commercially available library is used, hybridization may be performed according to the method described in the attached instructions. More preferably, hybridization is performed under a highly stringent condition.

The highly stringent condition mans that the sodium concentration is about 19 to 40 mM, preferably about 19 to 20 mM, and the temperature is about 50 to 70° C., preferably about 60 to 65° C.

The DNA having the base sequence represented by SEQ ID NO: 7 is used as the DNA encoding the partial peptide of the invention having the amino acid sequence represented by SEQ ID NO: 6; the DNA having the base sequence represented by SEQ ID NO: 9 is used as the DNA encoding the partial peptide of the invention having the amino acid sequence represented by SEQ ID NO: 8; the DNA having the base sequence represented by SEQ ID NO: 23 is used as the DNA encoding the partial peptide of the invention having the amino acid sequence represented by SEQ ID NO: 22; and the DNA having the base sequence represented by SEQ ID NO: 25 is used as the DNA encoding the partial peptide of the invention having the amino acid sequence represented by SEQ ID NO: 24.

In the cloning method of the DNA completely encoding the polypeptide of the invention or the partial peptide of the invention, the genome DNA and cDNA are amplified by the PCR method using a synthetic DNA primer comprising the partial base sequence of the polypeptide of the invention or the partial peptide of the invention; or DNAs (a library) assembled into appropriate vectors are selected by hybridization with DNA probes labeled with a radio isotope or an enzyme using DNA fragments of synthetic DNA encoding a part or entire region of the polypeptide of the invention or the partial peptide of the invention. Hybridization can be performed by the method known in the art, or according to the method, for example according to the method described in Molecular Cloning, 2nd edition (J. Sambrook et. al., Cold Spring Harbor Lab. Press, 1989). When a commercially available library is used, hybridization may be performed according to the method described in the attached instructions.

The base sequence of DNA is substituted using PCR or a kit known in the art, for example Mutan™-super Express Km (Takara Bio Inc.) and Mutan™-K (Takara Bio Inc.), by a method known in the art such as ODA-LA PCR method, Gapped duplex method, and Kunkel method, or according to the method.

DNAs encoding the cloned polypeptide may be directly used depending on the object, or may be used by digesting with a restriction enzyme, if desired, or by adding a linker. The DNA contains ATG as a translation initiation codon at the terminal, and may contain TAA, TGA or TAG as a translation termination codon at the other terminal. Such translation initiation codon and translation termination codon are added using an appropriate synthetic DNA adapter.

Expression vectors of the polypeptide of the invention and partial peptide of the invention can be manufactured by (A) excising the desired DNA fragment from the DNA (for example cDNA) comprising the DNA encoding the polypeptide or partial peptide of the invention, and (B) linking the DNA fragment at downstream of the promoter in an appropriate expression vector.

The vectors available include plasmids derived from Escherichia coli (for example pBR 322, pBR 325, pUC 12 and pUC 13), plasmids derived from Bacillus subtilis (for example pUB110, pTP5 and pC194), plasmids derived from yeast (for example pSH19 and pSH15), bacteriophages such as λ-phage, and animal viruses such as retrovirus, vaccinia virus and baculovirus, as well as pA1-11, pXT1, pRc/CMV, pRc/RSV and pcDNAI/Neo.

Any promoters may be used in the invention so long as they are appropriate promoters corresponding to hosts used for expression of genes. For example, they include SRα promoter, SV40 promoter, LTR promoter, CMV promoter, HSV-TK promoter and β-actin.

CMV (cytomegalovirus) promoter and SRa promoter are preferably used among them. Trp promoter, lac promoter, recA promoter, λP_{L} promoter, lpp promoter and T7 promoter are preferable when the host belongs to Escherichia sp., SPO1 promoter, PSO2 promoter and penP promoter are preferable when the host belongs to Bacillus sp, and PHO5 promoter, PGK promoter, GAP promoter and ADH promoter are preferable when the host belongs to yeast. Polyhedrin promoter and P10 promoter are preferable when the host is an insect cell.

Expression vectors comprising an enhancer, spricing signal, poly-A addition signal, selection marker and SC40 replication origin (may be referred to as SV40ori hereinafter) may be used, if desired, in addition to those described above. Examples of the selection marker include dehydrofolic acid reductase (abbreviated as dhfr hereinafter) gene [methotrexate (MTX) resistant], ampicillin resistant gene (abbreviated as Amp^{r} hereinafter), neomycin resistant gene (abbreviated as Neo^{r} hereinafter), and G418 (geneticin) resistant gene. When dhfr gene is used as the selection marker using dhfr deletion Chinese hamster cells, recombinant cells can be selected using a medium containing no thymidine.

A signal sequence suitable for a host is added, if necessary, to the N-terminal of the polypeptide of the invention. The signal sequences available are PhoA signal sequence and OmpA signal sequence when the host belongs to Escherichia sp., the signal sequence available are amylase signal sequance and subtilicin signal sequence when the host belongs to bacillus sp., the signal sequence available are MFα signal sequence and SUC2 signal sequence when the host belongs to yeast, and the signal sequence available are insulin signal sequence α-interferon signal sequence and antibody molecule signal sequence when the host belongs to animal cells.

Transformants can be produced using the vectors containing DNAs encoding the polypeptide of the invention or the partial peptide of the invention.

The hosts available include bacteria belonging to Escherichia sp., bacteria belonging to Bacillus sp., yeast, insect cells, insects and animal cells.

Examples of bacteria belonging to Escherichia sp. include Escherichia coli K12•DH1 (Proc. Natl. Acad. Sci. USA, vol. 60, 160, 1968), JM103 (Nucleic Acids Research, vol. 9, 309, 1981), JA221 (Journal of Molecular Biology, vol. 120, 517, 1978), HB101 (Journal of Molecular Biology, vol. 41, 459, 1969), and C600 (Genetics, vol. 39, 440, 1954).

Examples of bacteria belonging to Bacillus sp. include Bacillus subtilis (Gene, vol. 24, 255, 1988), and 207-21 (Journal of Biochemistry, vol. 95, 87, 1984).

Examples of yeast include Saccharomyces cerevisiae AH22, AH22R⁻, NA87-11A and DKD-5D, 20B-12, Schizosaccaromyces pombe NCYC 1913 and NCYC 2036, and Pichia pastoris KM71.

Examples of the insect cells available include established cell lines derived from the cells of larvae of Spodoptera frugiperda (Sf cells), MGI cells derived from middle intestine of Trichoplusia ni, High Five™ cells derived from eggs of Trichoplusia ni, cells derived from Mamestra brassicae, or cells derived from Estigmena acrea when the virus is AcNPV. The insect cells include established cell lines derived from Bombix mori N cells (BmN cells) when the virus is BmNPV. Examples of the Sf cells available include Sf9 cells (ATCC CRL 1711) and Sf21 cells described by Vaughn, J. L. et al (In Vivo, vol. 13, 213-217, 1977).

Larvae of silk worm are used as the insect (Maeda et. al., Nature, vol. 315, 592, 1985).

Examples of animal cells available include monkey cell COS-7 and Vero, Chinese hamster cell CHO (abbreviated as CHO hereinafter), dhfr gene defficient Chinese hamster cell CHO (abbreviated as CHO(dhfr⁻) hereinafter), mouse L cells, mouse AtT-20 cells, mouse myeloma cells, rat CH3 cells and human FL cells.

Bacteria belonging to Escherichia sp. can be transformed, for example, by the methods described in Proc. Natl. Acad. Sci. USA, vol. 69, 2110 (1972) and in Gene, vol. 17, 107 (1982).

Bacteria belonging to Bacillus sp. can be transformed, for example, by the method described in Molecular & General Genetics, vol. 168, 111 (1979).

Yeast can be transformed, for example, by the methods described in Methods in Enzymology, vol. 194, 182-187 (1991), and in Proc. Natl. Acad. Sci. USA, vol. 75, 1929 (1978).

Insects or insect cells can be transformed, for example, by the method described in Bio/Technology, vol. 6, 47-55 (1988).

Animal cells can be transformed, for example, by the method described in Cell Engineering Suppl. Vol. 8, New Protocol of Cell Engineering, 263-267 (1995) published by Shu-Jun-sha, and in Virology vol. 52, 456, 1973.

The transformants transformed with the expression vector comprising the DNA encoding the polypeptide can be obtained as described above.

When the transformants whose hosts are bacteria belonging to Escherichia sp. and Bacillus sp. are cultivated, a liquid medium is appropriate as the medium used for cultivation, which contains carbon sources nitrogen sources, minerals and other ingredients necessary for growth of the transformant. Examples of the carbon source include glucose, dextrin, soluble protein and saccharide, examples of the nitrogen source include organic and inorganic substances such as ammonium salts, nitrate salts, corn steep liquor, peptone, casein, yeast extracts, meat extracts, soybean flakes and potato extracts, and examples of minerals include calcium chloride, sodium dihydrogen phosphate and magnesium chloride. Yeast extracts, vitamins and growth acceleration factors may be also added. The desirable pH of the medium is 5 to 8.

An example of the culture medium for cultivating the bacteria belonging to Escherichia sp. is preferably M9 medium containing glucose and casamino acid (Miller, Journal of Experiments in Molecular Genetics, 431-433, Cold Spring Harbor Laboratory, New York, 972). For permitting the promoter to efficiently work, an agent such as 3β-indoacrylic acid may be added in the medium.

When the host is bacteria belonging to Escherichia sp., the host is usually cultivated at 15 to 43° C. for about 3 to 24 hours with aeration and stirring, if necessary.

When the host is bacteria belonging to bacillus sp., the host is usually cultivated at about 30 to 40° C. for about 6 to 24 hours with aeration and stirring, if necessary.

Examples of culture media for cultivating transformants when the host is yeast include Burkholder minimum medium (Bostian, K. L. et. al., Proc. Natl. Acad. Sci. USA, vol. 77, 4505, 1980), and SD medium containing 0.5% of casamino acid (Proc. Natl. Acad. Sci. USA, vol. 81, 5330, 1984). The pH value of the medium is preferably adjusted to 5 to 8. The host is cultvated at 20 to 35° C. for 24 to 72 hours with aeration and stirring, if necessary.

For cultivating the transformant when the host is insect cells, the preferable culture medium used is Grace's insect medium (Grace, T. T. C., Nature, vol. 195, 788, 1962) appropriately containing 10% inactivated bovine serum. The pH of the medium is preferably adjusted to 6.2 to 6.4, and the host is cultivated at 20 to 35° C. for 24 to 72 hours with aeration and stirring, if necessary.

Examples of the preferable culture medium for cultivating the transformant when the host is animal cells include MEM medium containing 5 to 20% of bovine fatus serum albumin, (Science, vol. 122, 501, 1952), DMEM medium (Virology, vol. 8, 396, 1959), RPMI 1640 medium (The Journal of the American Medical association, vol. 199, 519, 1967), and 199 medium (Proceeding of the Society for the Biological Medicine, vol. 73, 11, 1950). The pH of the medium is preferably adjusted to 6 to 8, and the host is cultivated at 30 to 40° C. for 15 to 60 hours with aeration and stirring, if necessary.

The polypeptide of the invention, or the partial peptide of the invention can be formed within the cells, within the cell membranes or out of the cells (preferably out of the cells) as described above.

The polypeptide of the invention, or the partial peptide of the invention can be isolated and purified from the cultivation product by the following methods.

For extracting the polypeptide of the invention, or the partial peptide of the invention from the cultured bacteria or cells, the cells are collected by a method known in the art after cultivation, and the cells are suspended in a buffer solution to destroy them by ultrasonic wave, using lysozyme and/or by freezing and melting, followed by obtaining a crude extract of the polypeptide by centrifugation or filtration. A protein denaturing agent such as urea and guanidine hydrochloride, and a surfactant such as Triton X-100™ may be contained in the buffer solution. When the polypeptide is secreted in the culture medium, the bacteria cells or other cells are separated from the supernatant by a method known in the art, and the supernatant is collected.

The supernatant obtained as described above, or the polypeptide of the invention or the partial peptide of the invention can be purified by an appropriate combination of the separation and purification methods known in the art. In these separation and purification methods known in the art, the difference of solubility is utilized in salting-out and solvent precipitation methods, the difference of molecular weight is mainly utilized in dialysis, ultra-filtration, gel permeation and SDS-polyacrylamide gel electrophoresis, the difference of charge is utilized in ion-exchange chromatography, specific affinity is utilized in affinity chromatography, the difference of hydrophobicity is utilized in reversed phase high performance liquid chromatography, and the difference of isoelectric points is utilized in isoelectric point electrophoresis.

When the polypeptide of the invention or the partial peptide of the invention is obtained as a free peptide, it can be converted into a salt by a conventional method or according to the conventional method. When the polypeptide of the invention or the partial peptide of the invention is obtained as a slat, on the contrary, it can be converted into a free peptide or another salt by a conventional method or according the conventional method.

The polypeptide of the invention or the partial peptide of the invention can be arbitrarily modified, or the polypeptide of the invention or the partial peptide of the invention can be partially eliminated by allowing an appropriate protein modification enzyme or protein decomposition enzyme to react before or after purification. Examples of such enzymes available include trypsin, chymotrypsin, arginine endopeptidase, protein kinase and glycosidase.

The presence of the polypeptide of the invention or the partial peptide of the invention can be detected by enzyme immunoassay or Western blot analysis using specific antibodies.

It is also possible to detect the presence of the polypeptide of the invention or the partial peptide of the invention by allowing an any exogenous peptide sequence that may serve as an epitope (antigen recognition site) to fuse to the N- or C-terminal of the polypeptide of the invention or the partial peptide of the invention (for example FLAG, His tag, myc tag, HA tag or HSV tag), and by detecting chemical luminescence using an antibody recognizing the peptide sequence. For example, the polypeptide comprising the amino acid sequence represented by SEQ ID NO: 16 is prepare, and the presence of the polypeptide of the invention or the partial peptide of the invention is detected by the method in Example 4 to be described hereinafter, or by the method similar to the method above.

The antibody against the polypeptide of the invention or the partial peptide of the invention (referred to as the antibody of the invention hereinafter) may be either a polyclonal antibody (referred to as the polyclonal antibody of the invention hereinafter) or a monoclonal antibody (referred to as the monoclonal antibody of the invention hereinafter), so long as it is able to recognize the polypeptide of the invention or the partial peptide of the invention.

The antibody against the polypeptide of the invention or the partial peptide of the invention can be produced using the polypeptide of the invention or the partial peptide of the invention as an antigen according to the method for producing the antibody or an anti-serum known in the art.

[Preparation of Monoclonal Antibody]

(a) Preparation of Monoclonal Antibody Producing Cell

The polypeptide of the invention or the partial peptide of the invention is administered, alone or together with a carrier and diluent, to a warm-blooded animal to a site capable of producing the antibody. A perfect Freund's adjuvant or an imperfect Freund's adjuvant may be administered together for enhancing antibody producing ability. The peptide is administered once for every 2 to 6 weeks by 2 to 10 with total number of challenges of 2 to 10. While the warm-blooded animal used is monkey, rabbit, dog, guinea pig, mouse, rat, sheep, goat or avian, mouse and rat are preferably used.

For preparing the monoclonal antibody producing cells, individuals manifesting a certain amount of antibody titer are selected from the warm-blooded animal, for example mouse, immunized with the antigen, the spleen or lymph node is sampled 2 to 5 day after the final immunization, and the antibody producing cells are fused with myeloma cells of the same or a different species of the animal to prepare a monoclonal antibody producing hybridoma. The antibody titer in the antiserum can be determined, for example, by measuring the activity of the label bound to the antibody after allowing the labeled polypeptide to react with the method by Keller and Milstein (Nature, vol. 256, 495, 1975). While polyethylene glycol (PEG) or Sendai virus is used as the fusion accelerating agent, PEG is preferable.

While examples of the myeloma cells include those of the warm-blooded animals such as NS-1, P3U1, SP2/0 and AP-1, P3U1 is preferably used. The preferable ratio of the number of the antibody producing cells (spleen cells) to the number of the myeloma cells is 1:1 to 20:1, the concentration of PEG (preferably PEG 1000 to PEG 6000) added is 10 to 80%, and the cells are efficiently fused by incubating at 20 to 40° C., preferably at 30 to 37° C., for 1 to 10 minutes.

While the monoclonal antibody producing hybridoma is screened by various methods, an example of the method comprises adding the supernatant of the hybridoma culture medium to a solid phase (for example a micro-plate) on which the polypeptide antigen is adsorbed directly or together with a carrier; adding an anti-immunoglobulin antibody (anti-mouse immunoglobulin antibody is used when the cells used for cell fusion are derived from mouse) or protein A each labeled with a radioactive substance or an enzyme; and detecting the monoclonal antibody immobilized on the solid phase. Alternatively, the method comprises adding the supernatant of the hybridoma culture medium to a solid phase on which the anti-immunoglobulin antibody or protein A is adsorbed, and detecting the monoclonal antibody immobilized on the solid phase.

The monoclonal antibody may be selected by a method known in the art, or by the method similar to the method known in the art. Usually, the antibody is selected in a animal cell cultivation medium supplemented with HAT (hypoxanthine, aminopterin and thymidine). Any medium may be used as the selection and bleeding medium so long as the hybridoma is able to grow. For example, the media available include RPMI 1640 medium containing 1 to 20%, preferably 10 to 20% of bovine fetus serum or GIT medium containing 1 to 10% of bovine fatuous serum (made by Wako Pure Chemical Co.), or a hybridoma cultivation serum free medium (made by Nissui Pharmaceuticals Co.). The cultivation temperature is usually 20 to 40° C., preferably about 37° C. The cultivation time is usually 5 days to 3 weeks, preferably 1 to 2 weeks. The cells are cultivated in a 5% carbon dioxide atmosphere. The antibody titer of the supernatant of the hybridoma cultivation medium may be determined by the same method as measuring the antibody titer in the anti-serum.

(b) Purification of Monoclonal Antibody

The monoclonal antibody can be isolated and purified by a method known in the art, for example by the separation and purification method of immunoglobulin (for example salting-out, alcohol precipitation, isoelectric precipitation, absorption and desorption using an ion exchanger (for example DEAE), ultra-centrifugation, gel filtration, or specific purification by which only the antibody is collected using an active adsorbent such as protein A or protein G, followed by dissociating the bond to obtain the antibody).

[Preparation of Polyclonal Antibody]

The polyclonal antibody of the invention can be produced by the method known in the art or according to the method. For example, the polyclonal antibody may be produced by preparing an immunogen (polypeptide antigen) or a complex of the immunogen with a carrier protein, immunizing a warm-blooded animal by the same method as preparing the monoclonal antibody, collecting a substance containing an antibody against the polypeptide of the invention or partial peptide of the invention from the immunized animal followed by separation and purification of the antibody.

For preparing the complex of the immunogen to be used for immunizing the warm-blooded animal with the carrier protein, the kind of the carrier protein and the mixing ratio between the carrier and hapten may be arbitrarily selected so long as the antibody is efficiently produced by immunizing the hapten cross-linked with the carrier. However, carriers such as bovine serum albumin, bovine thyroglobulin or hemocyanin is coupled with the hapten in a weigh ratio of about 0.1 to 20, preferably about 1 to 5, relative to the hapten.

Examples of the condensation agent used for coupling between the hapten and carrier include glutaraldehyde, carbodiimide, maleimide active ester and an active ester containing a dithiopyridil group.

The condensation product itself or a mixture thereof with a carrier and diluent is administered to the warm-blooded animal at a site capable of producing the antibody. A perfect Freund's adjuvant or an imperfect Freund's adjuvant may be administered for enhancing antibody producing ability after administration. The antigen is administered once per every about 2 to 6 weeks with a total number of challenges of about 3 to 10.

The polyclonal antibody can be collected from the blood or ascites fluid of the warm-blooded animal immunized as described above.

The titer of the polyclonal antibody in the anti-serum can be determined by the same method as measuring the titer of the antibody in the anti-serum. The polyclonal antibody can be purified by the separation and purification method of immunoglobulin as used in separation and purification of the monoclonal antibody.

With respect to an antisense DNA having a complementary or substantially complementary base sequence with the DNA of the invention, any antisense DNA may be used so long as it involves a complementary or substantially complementary base sequence with the DNA of the invention, and has an action capable of suppressing expression of the DNA.

Examples of the base sequence substantially complementary to the DNA of the invention include those having 70% or more, preferably 80% or more, more preferably 90% or more, and most preferably 95% or more of homology with the total base sequence or partial base sequence of the base sequence (or the complementary strand of the DNA of the invention) of the DNA of the invention. It is particularly suitable that the antisense DNA has 70% or more, preferably 80% or more, more preferably 90% or more, and most preferably 95% or more of homology with the complementary strand of the base sequence (for example the base sequence in the vicinity of the initiation codon), of the total base sequence of the complementary strand of the DNA of the invention, at the portion encoding the N-terminal portion of the polypeptide of the invention. These antisense DNA can be produced using a DNA synthesizer known in the art.

When the polypeptide of the invention has a signal peptide, the polypeptide is efficiently secreted out of the cell, and is able to manifest an important biological activity for signal transfer and self-defense.

Use of the polypeptide of the invention and partial peptide of the invention (the polypeptide of the invention and partial peptide of the invention are collectively referred to as the polypeptide of the invention hereinafter), and use of the DNA of the invention, antibody of the invention and antisense DNA are described below.

(1) Physiologically active peptides/proteins are usually featured as follows:

1. They are secreted out of the cell after being synthesized in the cell;

2. They are initially formed as precursor proteins, specifically excised from the precursor proteins in the course of secretion, and converted into matured (active) molecular species; and

3. Expression of the active molecules is specifically distributed in the tissue.

Examples of gene products featured as described above include insulin, relaxin, endoserine, propiomelanocortin, CRF, GHRH, LH-RH, PrRP, somatostatin, adrenomedulin, galanin, tachykinin, VIP, PACAP, glucagon and motilin.

Accordingly, the polypeptide of the invention provided with the conditions above is a useful biologically active substance as the gene products above. The polypeptide can act on target tissues located at near the expression tissue in an autocrine manner or in a paracrine manner, or can affect to a remote target tissue by flowing into the blood. Consequently, the polypeptide is considered to play an important role for maintaining homeostasis of the body such as genesis, growth and protection from invasion.

(2) Since the polypeptide of the invention is specifically expressed in the tissues such as the trachea and fetus lung as will be apparent from the description in Example 2, it can be used as a marker in these tissues. In other words, the peptide is useful as a marker for detecting differentiation of the tissue, disease conditions and onset of cancers. It can be also used for fractionating corresponding receptors and competitive polypeptides, as well as for investigating biological activities as a panel for high through-put screening known in the art. Furthermore, the peptide can be utilized for researches of hereditary diseases using chromosome mapping.

(3) Therapeutic and preventive medicines of various diseases related to the polypeptide of the invention

Since the polypeptide of the invention exists as a humoral factor in the body, diseases such as respiratory organ diseases, cancers, immune diseases, infectious diseases, digestive tract diseases, circulatory organ diseases, internal secretion diseases, and bone and joint diseases may occur when the polypeptide of the invention or the DNA of the invention has some abnormalities or deletion, or when they are abnormally decreased or increased.

Accordingly, the polypeptide of the invention or the DNA of the invention may be used as therapeutic and preventive agent of various diseases such as respiratory organ diseases, cancers, immune diseases, infectious diseases, digestive tract diseases, circulatory organ diseases, internal secretion diseases, and bone and joint diseases.

For example, when transmittance of information in the cell is not sufficiently or normally exhibited in a patient due to decrease or deletion of the peptide of the invention, the role of the polypeptide of the invention in the patient can be sufficiently or normally exhibited by (A) administering the DNA of the invention to the patient for allowing the polypeptide to be expressed in the body, (B) transplanting the cells into which the DNA of the invention is introduced and in which the DNA of the invention is expressed to the patient suffering from the disease as described above, or (C) administering the polypeptide of the invention to the patient.

When the DNA of the invention is used as the therapeutic and preventive agent as described above, it can be administered, alone or after inserting into an appropriate vector such as a retrovirus vector, adenovirus vector, adenovirus associated virus vector, to a human or other warm-blooded animals by a conventional method. The DNA of the invention may be formulated alone or together with a physiologically acceptable carrier such as an auxiliary substance for acceleration of intake, and can be administered through a gene gun or catheter such as a hydrogel catheter.

Examples of additives that can be mixed in the tablet and capsule include binders such as gelatin, corn starch, tragacanth gum, and gum Arabic; excipients such as crystalline cellulose; swelling agents such as corn starch, gelatin and alginic acid; sweeteners such as sucrose, lactose and saccharin; and flavoring agents such as peppermint, Gaultheria oil and cherry oil. Liquid carriers such as oils or fats may be contained in the materials as described above, when the formulation is an capsule. Aseptic compositions for injection can be formulated according to usual formulation practice by which an active substance and naturally occurring oils such as sesame oil and perm oil are dissolved or suspended in a vehicle such as injection water.

Examples of aqueous solutions for injection include physiological saline solution and isotonic solution containing glucose and other auxiliary agents (for example D-sorbitol, D-mannitol and sodium chloride). The solution may be used together with an appropriate dissolution aids such as alcohol (for example ethanol), polyalcohol (for example propyleneglycol and polyethylene glycol), and nonionic surfactant (for example Polysorbate 80™ and HCO-50). Examples of oily liquids include sesame oil and soy bean oil, and they may be used to together with benzyl benzoate or benzyl alcohol as a dissolution aid. A buffer solution (for example phosphate or sodium acetate buffer), an analgesic (for example benzalkonium or procain hydrochloride), a preservative (for example benzyl alcohol or phenol), and an antioxidant may be blended to the formulation. The formulated injection solution is usually filled in an appropriate ampoule.

The vector in which the DNA of the invention is inserted is also formulated as described above, and is usually used as a non-oral preparation.

Since the formulation obtained as described above is safe and low toxic, it may be administered to mammals (for example human, rat, mouse, guinea pig, sheep, swine, bovine, horse, cat, dog and monkey).

Although the dosage of the polypeptide of the invention differs depending on the disease, administration object and administration route, it is about 1 mg to 1000 mg, preferably about 10 to 500 mg, and more preferably about 10 to 200 mg per day in an adult (body weight 60 kg) when the polypeptide of the invention is orally administered for therapy of the respiratory organ disease. While the dosage of the polypeptide of the invention differs depending on the administration object and disease when it is administered non-orally, it is favorable to administer the polypeptide of the invention in a dosage of about 1 to 1000 mg, preferably about 1 to 200 mg, and more preferably about 10 to 100 mg per one day when it is administered to an adult (body weight 60 kg) as an injection solution for therapy of the respiratory organ disease. The dosage may be converted into the dosage per 60 kg of the body weight when the polypeptide of the invention is administered to an animal other than human.

(4) Screening of Candidate of Medicine for Disease

Since the polypeptide of the invention is contained in vivo (particularly in the trachea and fetus lung) as a humoral factor, a compound for promoting the function of the polypeptide of the invention, and salts thereof, may be used, for example, as therapeutic and preventive medicines of diseases such as respiratory organ diseases, cancers, immune diseases, digestive tract diseases, circulatory organ diseases, internal secretion diseases and bone and joint diseases.

On the contrary, a compound for inhibiting the function of the polypeptide of the invention, and salts thereof, may be also used as therapeutic and preventive medicines of diseases caused by excess production of the polypeptide of the invention.

Accordingly, the polypeptide of the invention is useful as an agent for screening the compound or salts thereof that promotes or inhibit the function of the polypeptide of the invention.

The invention provides:

(1) a method for screening a compound or salts thereof that promote the function of the polypeptide of the invention or salts thereof (abbreviated as a promoter hereinafter), or a compound or salt thereof that inhibit the function of the polypeptide of the invention or salts thereof using the polypeptide of the invention or salts thereof.

The screening kit of the invention includes the polypeptide of the invention or salt thereof.

The compound or salts thereof obtained using the screening method or screening kit of the invention is a compound selected from peptides, proteins, non-peptide compounds, synthetic compounds, fermentation products, plant extracts, animal tissue extracts and plasma, and promotes or inhibits the function of the polypeptide of the invention.

The same salts the salts of the polypeptide of the invention are used as the salts of the compound.

The compound obtained by using the screening method or screening kit of the invention may be used as the therapeutic or preventive agent by conventional methods. For example, tablets, capsules, elixirs, microcapsules, aseptic solutions and dispersions may be prepared by the same method as used in the medicines containing the polypeptide of the invention.

Since the preparation obtained as described above is safe and low toxic, it can be administered to mammals (for example human, rat, mouse, guinea pig, sheep, swine, bovine, horse, cat, dog and monkey).

While the dosage of the compound or the salts thereof differs depending on the administration object, disease and administration route, it is about 0.1 to 100 mg, preferably about 1.0 to 50 mg, and more preferably about 1.0 to 20 mg per one day for an adult (body weight 60 kg) per one day when the compound for promoting the function of the polypeptide of the invention is orally administered for the therapy of the respiratory organ diseases. The compound is administered non-orally to an adult (body weight 60 kg) by intravenous injection for the therapy of the respiratory diseases, the dosage is favorably about 0.01 to 30 mg, preferably about 0.1 to 20 mg, and more preferably about 0.1 to 10 mg per one day, although the dosage differs depending on the object of administration and diseases. The dosage may be converted into the dosage per 60 kg of the body weight when the polypeptide of the invention is administered to an animal other than human.

When the compound for inhibiting the function of the polypeptide of the invention is orally administered, the dosage is about 0.1 to 100 mg, preferably about 1.0 to 50 mg, and more preferably about 1.0 to 20 mg per one day for an adult (body weight 60 kg). When the compound for inhibiting the function of the polypeptide of the invention is administered non-orally to an adult (body weight 60 kg) as an injection solution through intravenous injection, the dosage is favorably about 0.01 to 30 mg, preferably 0.1 to 20 mg, and more preferably about 0.1 to 10 mg per one day, although the dosage differs depending on the administration object and diseases. The dosage may be converted into the dosage per 60 kg of the body weight when the polypeptide of the invention is administered to an animal other than human.

(5) Quantitative Analysis of the Polypeptide of the Invention

Since the antibody of the invention is able to specifically recognize the polypeptide of the invention, it can be used for quantitative analysis, particularly for quantitative analysis by a sandwich immunoassay method, of the polypeptide of the invention in test samples.

The invention provides:

(1) a method for quantitatively analyzing the polypeptide of the invention comprising the steps of allowing the test solution to competitively react with the labeled polypeptide of the invention, and measuring the proportion of the labeled polypeptide of the invention bound to an antibody; and

(2) a method for quantitatively analyzing the polypeptide of the invention comprising the steps of allowing the test solution to simultaneously or continuously react with the antibody of the invention immobilized on a carrier and another labeled antibody of the invention, and measuring the activity of the label on the immobilizing carrier.

Quantitatively analysis as well as detection by tissue staining of the polypeptide of the invention is possible using the monoclonal antibody of the invention. Either the antibody molecule itself, or F(ab′)₂, Fab′ or Fab fraction may be used for these objects.

The qualitative analysis method of the polypeptide of the invention using the antibody of the invention is not particularly restricted. Any analysis methods may be used so long as the quantity of the antibody, antigen or antibody-antigen complex corresponding to the quantity of the antigen (for example the quantity of the polypeptide of the invention) in the test solution is determined by a chemical or physical method, and the quantity is calculated based on a calibration curve prepared using standard solutions containing a known quantities of the antigen. While nephelometry, competitive method, immunometric method and sandwich method are favorably used, the sandwich method to be described below is particularly preferable in view of sensitivity and specificity.

Labeling agents used for the measuring method using the label substance include radioactive isotopes, enzymes, fluorescent substances and luminescent substances. Examples of the radioactive isotopes include [¹²⁵I], [¹³¹I], [³H] and [¹⁴C]. Enzymes being stable and having a high specific activity are preferable as the enzymes above, and examples of them available include β-galactosidase, β-glucosidase, alkali phosphatase, peroxidase and malic acid dehydrogenase. Examples of the fluorescent substance available include fluorescamine, and fluorescent isothiocyanate. Examples of the luminous substance available include luminol, luminol derivatives, luciferin and lucigenin. In addition, biotin-adipin complexes may be used for binding the antibody or antigen and labeling agent.

Physical adsorption may be used for immobilizing the antigen or antibody, and a method using a chemical bond that is conventionally used for insolubilizing or immobilizing polypeptides or enzymes may be used. Examples of the carrier include insoluble polysaccharides such as agarose, dextran and cellulose, synthetic resins such as polystyrene, polyacrylamide and silicone, and glass.

The quantity of the polypeptide of the invention in the test solution can be qualitatively analyzed in the sandwich method by the steps comprising allowing the test solution to react with the immobilized monoclonal antibody of the invention (primary reaction), allowing another labeled monoclonal antibody of the invention (secondary reaction) to react, and measuring the activity of the labeling agent on the immobilizing carrier. The order of the primary and secondary reactions may be inverted or both reactions may be simultaneously proceeded, or the reactions may be carried out with a time lag. Similar labeling agents and similar immobilizing methods to those described above may be used. The antibody to be used for the solid phase antibody and labeled antibody are not always required to be one kind of the antibody, and a mixture of at least two kinds of the antibodies may be used for enhancing sensitivity.

Antibodies having different binding sites of the polypeptide of the invention are preferably used as the monoclonal antibodies of the invention to be used in the primary and secondary reactions, respectively, in the method for measuring the polypeptide of the invention by the sandwich method of the invention. This means that the antibody used in the primary reaction recognizes a site other than the C-terminal, for example the N-terminal, in the antibodies used in the primary and secondary reactions, respectively, when the antigen used in the secondary reaction recognizes the C-terminal of the polypeptide of the invention.

The monoclonal antibody of the invention can be used for measuring systems other than the sandwich method, for example for the competitive method, immunometric method or nephelometry.

In the competitive method, an unreacted labeled antigen (F) is separated from a labeled antigen (B) bonded to the antibody (B/F separation) after allowing the antigen and labeled antigen in the test solution to competitively react with the antibody; and either the quantity of the B or the quantity of F is measured to determine the quantity of the antigen in the test solution. Either a liquid phase method or a solid phase method is used for the reaction method of the invention, wherein a soluble antibody is used as the antibody, and polyethylene glycol is used for the B/F separation while using a second antibody in the liquid phase method; and the immobilized antibody or soluble antibody is used as the first antibody, and an immobilized antibody is used as the second antibody in the solid phase method.

In the immunometric method, the solid phase is separated from the liquid phase after allowing the antigen in the test solution and immobilized antigen to competitively react with a given amount of the labeled antibody; or the solid phase is separated from the liquid phase after allowing the antigen in the test solution to react with an excess amount of the labeled antibody followed by allowing the unreacted labeled antibody to bind to the solid phase by adding the immobilized antigen. Subsequently, the quantity of the labeled antibody in any of the two phases is measured to quantitatively analyze the amount of the antigen in the test solution.

In nephelometry, the quantity of insoluble precipitates formed in the gel or solution as a result of the antigen-antibody reaction is measured. When the amount of the antigen in the test solution is very small to afford a minute quantity of the precipitate, laser nephelometry taking advantage of laser scattering may be favorably used.

Special conditions and operations are not needed for applying these immunological measuring method to the quantitative analysis method of the invention. The measuring system of the polypeptide of the invention may be constructed by adding technical considerations commonly known to those skilled in the art to the conventional conditions and operation methods in each measuring method. Details of these conventional technical means can be referenced to reviews and published articles.

Examples of the references are: “Radio-immunoassay”, ed. by Hirosi Irie, Kodan-sha, 1974; “Radio-immunoassay, continued edition”, ed. by Hirosi Irie, Kodansha, 1979; “Enzyme Immunoassay”, Eiji Ishikawa et. al., ed., Igaku Shoin, 1978; “Enzyme Immunoassay, 2nd edition”, Eiji Ishikawa et. al., ed., Igaku Shoin, 1982; and “Enzyme Immunoassay, 3rd edition”, Eiji Ishikawa et. al., ed., Igaku Shoin, 1987. The other references are: “Method in Enzymology, Vol. 70 (Immunochemical Techniques (part A)”; “Method in Enzymology, Vol. 73 (Immunochemical Techniques (part B)”; “Method in Enzymology, Vol. 74 (Immunochemical Techniques (part C)”; “Method in Enzymology, Vol. 84 (Immunochemical Techniques (part D: Selected Immunoassay)”; “Method in Enzymology, Vol. 92 (Immunochemical Techniques (part E: Monoclonal Antibodies and General Immunoassay Methods)”; and “Method in Enzymology, Vol. 121 (Immunochemical Techniques (part I: Hybridoma Technology and Monoclonal Antibodies”, all of which are published by Academic Press, Inc.

The polypeptide of the invention can be quantitatively analyzed with good sensitivity using the antibody of the invention.

Furthermore, the patient may be diagnosed to be suffering from a respiratory organ disease, cancer, immune disease, infectious disease, digestive tract disease, circulatory organ disease, internal secretion disease, or bone and joint disease, or has a high risk of suffering from one of these diseases, when an increase or a decrease of the concentration of the polypeptide of the invention has been detected by measuring the concentration of the polypeptide of the invention using the antigen of the invention.

The antibody of the invention can be also used for detecting the polypeptide of the invention in the test sample such as body fluid or tissues. In addition, the antibody of the invention can be used for preparing an antibody column for purifying the polypeptide of the invention, for detecting the polypeptide of the invention for fractionation in the purification process, and for analyzing the behavior of the polypeptide of the invention in the test cells.

(6) Gene Diagnosis Agent

Since abnormalities of DNA or mRNA (abnormalities of gene) encoding the polypeptide of the invention in mammals (for example, human, rat, mouse, guinea pig, rabbit, sheep, swine, bovine, horse, cat, dog and monkey) can be detected by using the DNA of the invention as a probe, it is useful as a gene diagnosis agent for damages of the DNA and mRNA, mutagenesis, depressed expression or over-expression.

The gene diagnosis using the DNA of the invention can be executed by the Northern hybridization and PCR-SSCP methods known in the art (Genomics, vol. 5, p874-879, 1989; Proceedings of the National Academy of Sciences of the United State of America, vol. 86, p2766-2770, 1989).

For example, when depressed expression is detected by Northern hybridization, or when mutation of DNA is detected by the PCR-SSCP method, the patient is diagnosed to be in high risk of onset of diseases such as respiratory organ disease, cancer, immune disease, infectious disease, digestive tract disease, circulatory organ disease, internal secretion disease, or bone and joint disease.

(7) Medicine Containing Antisense DNA

Since the antisense DNA that forms complimentary bonds with the DNA of the invention and suppresses expression of the DNA is able to depress the function of the polypeptide of the invention or the function of the DNA of the invention in the body, the antisense DNA can be used as a therapeutic and preventive agent of diseases ascribed to over-expression of the polypeptide of the invention.

The antisense DNA can be also used as the therapeutic and preventive agent by the same manner as the therapeutic and preventive agent of various diseases containing the DNA of the invention.

For example, the antisense DNA, alone or after inserting into an appropriate vector such as a retrovirus vector, an adenovirus vector or an adenovirus associated virus vector, may be used according to a conventional method. The antisense DNA is formulated by itself or together with a pharmaceutically acceptable carrier such as an auxiliary agent for promoting intake of the medicine, and is administered with a gene gun or through a catheter such as a hydrogel catheter.

In addition, the antisense DNA of the invention may be also used as a diagnosis oligonucleotide probe for investigating the presence and expression of the DNA of the invention in the tissue and cells.

(8) Medicine Containing the Antibody of the Invention

The antibody having a function for neutralizing the activity of the polypeptide of the invention can be used, for example, as a therapeutic and preventive agent of diseases ascribed to over-expression of the polypeptide of the invention.

The therapeutic and preventive agent of the diseases containing the antibody of the invention can be orally or non-orally administered, directly as a liquid medicine or by formulating into an appropriate formulation, to mammals (for example human, rat, rabbit, sheep, swine, bovine, cat, dog and monkey). While the dosage is different depending on the administration object, diseases, disease conditions and administration route, favorable dosage is usually 0.01 to 20 mg, preferably 0.1 to 10 mg, and more preferably 0.1 to 5 mg per kg body weight preferably 1 to 3 times per day by intravenous injection. Approximately the same dosage of oral or non-oral administration is possible. The dosage may be increased depending on the disease conditions, particularly when the condition is severe.

The antibody of the invention may be administered alone or as an appropriate composition. The composition contains a salt of the antibody, and a pharmaceutically acceptable carrier, diluent or excipient. Such composition is provided as a drug suitable for oral or non-oral administration.

Examples of the composition for oral administration include solid or liquid drugs such as tablets (including sugarcoated and film coated pills), pellets, granules, powders, capsules (including soft capsules), syrups, emulsions and dispersions. Such composition is manufactured by the method known in the art, and contains carriers, diluent or excipients conventionally used in formulation. The excipient includes galactose, starch, sucrose and magnesium stearate.

Compositions for non-oral administration are used, for example, for injection agents and suppositories, and the injection agent comprises drugs for hypodermic injection, intradermal injection, intramuscular injection and intravenous feeding. Such injection agents are prepared by the method known in the art, for example by dissolving, dispersing or emulsifying the antibody or a salt thereof in an asceptic aqueous or oily solution usually used for injection. Examples of the aqueous solution available for injection include a physiological saline solution and isotonic solutions containing glucose and other auxiliary agents, and appropriate solubilizing agents such as alcohols (such as ethanol), polyalcohols (such as propyleneglycol or polyethylene glycol) and nonionic surfactants [such as polysorbate 80 or HCO-50 (a polyoxyethylene (50 mol) adduct of hydrated castor oil)] may be used together. Examples of the oily solution available include sesame oil and soy bean oil, and benzyl benzoate or benzyl alcohol may be used together as a solubilizing agent. The injection solution prepared is usually filled in an appropriate ampoule. The suppository used for intrarectum administration can be prepared by mixing the antigen or a salt thereof with a suppository base.

The pharmaceutical composition for oral or non-oral administration is desirably formulated as a drug for a single dosage suitable for administering the active ingredient. Examples of such single dosage formulation include tablets, pills, capsules, injection agents (ampoules) and suppositories, and the dosage is 5 to 500 mg, particularly 5 to 100 mg in the injection agent and 10 to 250 mg in other formulations.

The composition above may contain other active ingredients so long as they do not cause unfavorable interactions with the antibody by blending.

(9) DNA Transgenic Animal

The invention provides non-human mammals having the exogenous DNA encoding the polypeptide of the invention (abbreviated as exogenous DNA of the invention hereinafter) or a mutated DNA thereof (abbreviated as the exogenous mutated DNA of the invention hereinafter).

The present invention provides:

(1) Non-human mammals having the exogenous DNA of the invention or a mutated DNA thereof;

(2) The animal according to (1), wherein the non-human mammal is a rodent;

(3) The animal according to (2), wherein the rodent is a mouse or rat; and

(4) A recombinant vector comprising the exogenous DNA of the invention or mutated DNA thereof, and being able to be expressed in the mammal.

The non-human mammals having the exogenous DNA of the invention or a mutated DNA thereof (abbreviated as the DNA transgenic animal of the invention hereinafter) may be created by introducing the desired DNA to fertilized ova, non-fertilized ova, sperms or embryonic cells including primordial germ cells thereof at the embryogenesis stage of the development of the mammals (more preferably at the single cell stage or fertilized ovum stage and before, in general, cell division to eight cells) by a calcium phosphate method, electrical pulse method, lipofection method, aggregation method, micro-injection method, particle gun method or DEAE-dextran method. The desired exogenous DNA of the invention is introduced into somatic cells, organs in the body or tissue cells to utilize the cells for cell cultivation and tissue culture. Alternatively, the DNA transgenic animal of the invention may be created by fusing these cells with embryonic cells by a cell fusion method known in the art.

Examples of the non-human mammals available include bovine, swine, sheep, goat, rabbit, dog, cat, guinea pig, hamster, mouse and rat. Rodents such as mice (for example pure lines such as C57BL/6 and DBA2 lines, and cross-bred lines such as B6C3F₁, BDF₁, B6D2F₁, BALC/c, ICR lines and the like) or rats (Wistar, S D and the like) are preferable among them, since these animals have a relatively short development and life cycle of individuals and are easy for breeding considering creation of disease model animals.

The “mammal” as an object of the recombinant vector being able to be expressed in the mammal include human in addition to the non-human mammals.

The exogenous DNA of the invention is not the DNA of the invention inherently involved in the non-human mammals, and means the DNA of the invention that has been once extracted and isolated from the mammal.

The mutated DNA of the invention includes the original DNA of the invention in which some abnormalities (for example mutation) such as addition, deletion or substitution with other bases are generated in the base sequence, as well as abnormal DNA.

The abnormal DNA means a DNA that expresses an abnormal polypeptide of the invention, for example a DNA that expresses a polypeptide that suppresses the function of the normal polypeptide of the invention.

The exogenous DNA of the invention may be derived from either the same as or different kind of animals from the animal as an object of the invention. It is usually advantageous to use a DNA construct linked at the downstream of the promoter capable of expressing the DNA in animal cells when the DNA of the invention is introduced into the desired animal. For example, when the human DNA of the invention is introduced, the DNA construct (for example a vector), which is formed by linking the human DNA of the invention at the downstream of various promoters that are able to express the DNAs derived from various mammals (for example rabbit, canine, cat, guinea pig, hamster, rat and mouse) having the DNAs highly homologous to the human DNA, is introduced into the fertilized ova, for example fertilized ova of mouse, by micro-injection in order to create a DNA introduced mammal capable of highly expressing the DNA of the invention.

Preferably used expression vectors of the polypeptide of the invention include the plasmid derived from Escherichia coli, the plasmid derived from Bacillus subtilis, plasmid derived from yeast, bacteriophages such as α-phage, and animal viruses including retroviruses such as Moloney leukemia virus, vaccinia virus and baculovirus. The plasmids derived from Escherichia coli, Bacillus subtilis and yeast are preferably used among them.

Examples of the promoters available for regulating expression of the DNA above include (1) DNA promoters derived from viruses (for example simian virus, cytomegalovirus, Moloney leukemia virus, JC virus, breast cancer virus and polio virus), and (2) promoters derived from various mammals (human, rabbit, canine, cat, guinea pig, hamster, rat and mouse), for examples promoters of albumin, insulin II, uroplakin II, esterase, erythropoietin, endothelin, muscle creatine kinase, glia fiber acidic protein, glutathione S transferase, platelet derived growth factor β, keratin K1, K10 and K14, collagen I and II, cyclic AMP dependent kinase p 1 subunit, dystrophin, tartaric acid resistant alkali phosphatase, atrium sodium diuresis factor, endothelial receptor tyrosine kinase (usually abbreviated as Tie2), sodium potassium adenosine-3-phosphatase (Na, K-ATPase), neurofilament light chain, metallothionein I and IIA, metalloproteinase I tissue inhibitor, MHC class I antigen (H-2L), H-ras, renin, dopamine-β-hydroxynase, thyroid gland peroxidase (TPO), polypeptide chain elongation factor 1α (EF-1α), β-actin, α and β myosin, myosin light chain 1 and 2, myelin base protein, thyroblobulin, Thy-1, immunoglobulin, H-chain variable part (VNP), serum amyloid component, myoglobin, troponin C, smooth muscle α-actin, preproenkepharin A, and vasopressin. The cytomegalovirus promoter, human polypeptide chain elongation factor 1α (EF-1α) promoter, and human and avian β-actin promoter that can be expressed in high densities in the entire body are favorable among them.

The vectors preferably have sequences that terminate translation of desired mRNAs in the DNA introduced animals (usually referred to as terminators). Examples of the sequence available include DNA sequences derived from viruses and various mammals, preferably simian virus SV40 terminator.

Spricing signals, enhancer regions and a part of introns of eukaryotic cell DNA may be linked between the promoter region and translation region, or at the 3′-downstream of the translation region in order to permit higher expression of the desired exogenous DNA.

The translation region can be prepared as the DNA construct capable of being expressed by conventional DNA engineering by which DNAs are linked at the downstream of the promoter and at the upstream of the translation termination site, if desired.

The exogenous DNA of the invention is introduced at the fertilized ovum stage so that the DNA is distributed in all the embryonic cells and somatic cells of the desired mammals. Existence of the exogenous DNA of the invention in the embryonic cells of the created animals after introducing the DNA means that the exogenous DNA of the invention is retained in all the embryonic cells and somatic cells in all the descendants of the created animals. The descendants of this sort of animals inheriting the exogenous DNA of the invention possess the exogenous DNA of the invention in all the embryonic cells and somatic cells thereof.

The non-human mammals into which the exogenous normal DNA of the invention has been introduced can be sub-bred in a normal breeding environment after confirming that the exogenous DNA is stably retained by cross-bleeding.

The exogenous DNA of the invention is introduced in the fertilized ovum stage so that the DNA is distributed in excess in all the embryonic cells and somatic cells of the desired mammals. Existence of an excess amount of the exogenous DNA of the invention in the embryonic cells of the created animals after introducing the DNA means that the descendants of the created animals possess an excess amount of the exogenous DNA of the invention in all the embryonic cells and somatic cells thereof. The descendants of this sort of animals inheriting the exogenous DNA of the invention has an excess amount of the exogenous DNA of the invention in all the embryonic cells and somatic cells thereof.

Homozygotes having the introduced DNA in both homologous chromosomes are obtained, and the animals are sub-bred by cross-breeding the male and female animals so that all the descendants possess an excess amount of this DNA.

The normal DNA of the invention is highly expressed in the non-human mammal having the normal DNA of the invention. Since hyperactivity of the polypeptide of the invention finally occurs by enhancing the function of normal intrinsic DNAs, the mammal can be utilized as the disease model animal of hyperactivity. For example, it is possible to elucidate the mechanism of hyperactivity of the polypeptide of the invention and diseases related to the polypeptide of the invention, and to investigate the therapeutic methods of these diseases using the animals into which the normal DNA of the invention has been introduced.

Since the mammal into which the exogenous normal DNA has been introduced manifests a symptom of increase of the free peptide of the invention, the mammal is applicable to a screening test of remedies against the diseases related to the polypeptide of the invention.

On the other hand, the non-human mammal possessing the exogenous abnormal DNA of the invention can be sub-bred in a usual breeding environment as the mammal possessing the corresponding DNA after confirming that the exogenous DNA is stably maintained by breeding. The desired exogenous DNA can be used as a DNA source by integrating it into a plasmid. A DNA construct with a promoter can be manufactured by conventional DNA engineering. The abnormal DNA of the invention is secured so as to be distributed in all the embryonic cells and somatic cells of the desired mammal by introducing the abnormal DNA of the invention at the fertilized ovum stage. Existence of the abnormal DNA of the invention in the embryonic cells of the created animal after introducing the DNA means that all the descendants of the created animal possess the abnormal DNA of the invention in all the embryonic cells and somatic cells thereof. The descendants of the animal inheriting the exogenous DNA of the invention possess the abnormal DNA in all the embryonic cells and somatic cells thereof. Homozygotes having the introduced DNA in both homologous chromosomes are obtained, and the animals are sub-bred by cross-breeding the male and female animals so that all the descendants possess this DNA.

The abnormal DNA of the invention is highly expressed in the non-human mammal possessing the abnormal DNA of the invention. Since a refractory symptom due to inactivation of the polypeptide of the invention finally occurs by inhibiting the function of the intrinsic normal DNAs, the animal can be utilized as a disease model of DNA function inactivation. For example, elucidation of the disease mechanism the refractory symptom due to inactivation of the polypeptide of the invention, and investigation of the therapeutic method of this disease are possible using the animal into which the abnormal DNA of the invention has been introduced.

In an practical example, the animal highly expressing the abnormal DNA of the invention may serve as a model for elucidating functional inhibition (dominant negative action) of the normal polypeptide by the abnormal polypeptide of the invention in the refractory symptom due to inactivation of the polypeptide of the invention.

Since the mammal into which the exogenous abnormal DNA has been introduced manifests a symptom of increase of the free peptide of the invention, the mammal is applicable to a screening test of remedies against the diseases related to the refractory symptom due to inactivation of the polypeptide of the invention.

Other possibilities of use of the two kinds of the animals into which the DNA of the invention has been introduced comprise:

[1] Use as cell sources for tissue culture;

[2] Analysis of genes and polypeptides that are specifically expressed, activated or inactivated by the polypeptide of the invention by comparative analysis of the DNA or RNA in the tissue of the animal into which the DNA of the invention has been introduced with the animals (control group) into which the DNA has not been introduced, or by comparative analysis of the polypeptide composition;

[3] Studies of cell functions from tissues that are difficult to be cultivated by cultivating the tissue cells containing the DNA of the invention by a standard tissue culture technique;

[4] Screening of medicines that enhance cell functions using the cells described in [3] above; and

[5] Isolation and purification of the abnormal polypeptide of the invention, and preparation of antibodies against the polypeptide.

Clinical conditions of the diseases related to the polypeptide of the invention including the refractory symptom due to inactivation of the polypeptide of the invention may be investigated using the animal into which the DNA of the invention has been introduced. Detailed pathological knowledge of organs in the disease model related to the polypeptide of the invention may be also obtained, which contributes to research and therapy of secondary diseases caused by the disease.

Cells into which free DNAs are introduced may be obtained using polypeptide (protein) degradation enzymes such as trypsin after extracting the organs from the animals into which the DNA of the invention has been introduced and mincing the organs. The cells can be cultivated or cloned. Furthermore, identification of the cells producing the polypeptide of the invention, relations with apoptosis, differentiation or proliferation, signal transfer mechanisms among the cells, and abnormalities of the signal transfer mechanism may be investigated to offer effective research materials for the polypeptide of the invention and for elucidation of the action thereof.

It is possible to provide an effective and rapid screening method of the remedies of these diseases using the test methods and quantification methods as described above using the animal into which the DNA of the invention has been introduced, in order to develop the remedies of the diseases related to the polypeptide of the invention including the refractory symptom due to inactivation of the polypeptide of the invention. It is also possible to investigate and develop DNA therapies of the diseases related to the polypeptide of the invention using the animal into which the DNA of the invention has been introduced, or a vector expressing the exogenous DNA of the invention.

(10) Knockout Animal

The invention provides embryonic stem cells of the non-human mammals in which the DNA of the invention is inactivated, and a non-human mammal in which expression of the DNA of the invention is deficient.

The invention provides:

(1) Non-human mammal embryonic stem cells in which the DNA of the invention is inactivated;

(2) The embryonic stem cells according to (1) in which the DNA of the invention is inactivated by introducing a reporter gene (for example, β-galactosidase gene derived from Escherichia coli) into the DNA;

(3) The embryonic stem cells according to (1) as a neomycin resistant cells;

(4) The embryonic stem cells according to (1), wherein the non-human mammal is a rodent;

(5) The embryonic stem cells according to (4), wherein the rodent is a mouse;

(6) A non-human mammal deficient in expression of the DNA, wherein the DNA of the invention is inactivated;

(7) The non-human mammal according to (6), wherein the DNA is inactivated by introducing a reporter gene (for example β-galactosidase gene derived from Escherichia coli), and the reporter gene is able to be expressed under the regulation of a promoter against the DNA of the invention;

(8) The non-human mammal according to (6), wherein the non-human mammal is a rodent;

(9) The non-human mammal according to (8), wherein the rodent is a mouse; and

(10) A method for screening a compound or a salt thereof for promoting or inhibiting the activity of the DNA of the invention comprising the steps of administering a test compound to the animal according to (7), and detecting expression of a reporter gene.

The non-human mammal embryonic stem cell in which the DNA of the invention is inactivated means the embryonic stem cells (abbreviated as ES cells hereinafter) of the non-human mammal in which the DNA substantially has no expression ability of the polypeptide of the invention (referred to as knockout DNA of the invention) by suppressing DNA expression ability or by substantially eliminating the activity of the polypeptide of the invention encoded by the DNA as a result of applying an artificial mutation to the DNA of the invention involved in the non-human mammal.

The non-human mammal used is the same as described previously.

Examples of the methods for allowing the DNA of the invention to be artificially mutated include deletion of a part of or all the DNA sequence, and insertion or substitution of other DNAs by gene engineering. The knockout DNA of the invention may be prepared by displacing codon reading frames, or by destroying the functions of promoters or exons.

The non-human mammal embryonic stem cells in which the DNA of the invention is inactivated (abbreviated as DNA inactivated ES cells of the invention or knockout ES cell of the invention hereinafter) can be obtained, for example, by the steps comprising isolating the DNA of the invention contained in the desired non-human mammal; disabling synthesis of perfect mRNAs by destroying the function of exons by inserting a drug resistant gene represented by a neomycin resistant gene and hygromycin resistant gene, or a reporter gene represented by a lacZ (β-galactosidase gene), cat (chloramphenicol acetyltransferase gene) into exon portions, or by inserting a DNA sequence (for example a poly-A addition signal) for terminating translation of the gene into the intron portion between the exons; introducing the DNA chain constructed so as to destroy the gene as a result of insertion above (abbreviated as a targeting vector hereinafter) into the chromosome of the animal by a homologous recombination method; analyzing the ES cells obtained by a Southern hybridization analysis using the DNA sequence on or in the vicinity of the DNA of the invention as a probe, or by a PCR method using the DNA sequence on the targeting vector and the DNA sequence in the vicinity of a region except the DNA of the invention used for preparing the targeting vector as primers; and selecting the knockout ES cells of the invention.

Established cell lines as described above may be used as the original ES cells before inactivating the DNA of the invention by a homologous recombination method, or new cell lines may be established according to the Evans and Kaufman method known in the art. For example, the 129 line ES cells are usually used today as the mouse ES cells. However, since the immunological background is not clear in the 129 ES cell line, cell lines established using BDF₁ (F₁ of C57BL/6 and DBA/2) mouse, in which a small level of ovulation of (C57BL/6 mouse and C57BL/6) is improved by cross-breeding with DBA/2 mouse, may be favorably used in place of the 129 ES cell line for the purpose of obtaining ES cells from a pure line whose genetic background is immunologically clear. The BDF1 mouse is advantageous due to its high level of ovulation in addition to toughness of eggs. Moreover, since the BDF1 mouse is a descendant of the C57BL/6 mouse, the genetic background the ES cells obtained using the BDF1 mouse may be traced to that of the C57ML/6 mouse by back crossbreeding with the C57BL/6 mouse when a disease model mouse is created using the ES cells.

While blastocysts 3 to 5 days after fertilization have been usually used for establishing the ES cells, many initial stage embryos may be efficiently obtained by collecting eight-cell stage embryos and cultivating to the blastocysts.

While either male or female ES cells may be used, the male ES cells are usually advantageous for creating regenerative chimera lines. It is desirable to discriminate the ES cells to be female or male as soon as possible in order to save the labor of cultivation.

An example the method for discriminating male or female of the ES cells is to amplify and detect the gene at the sex determining region on the Y-chromosome by PCR. Since the number of the ES cells in only one colony (about 50 cells) is sufficient for the karyotype analysis in contrast to the conventional method requiring about 10⁶ cells, discrimination of male or female becomes possible at the primary selection of the ES cells at the early stage of cultivation. Selection of male cells at the early stage permits the labor of initial cultivation to be largely reduced.

The number of the chromosomes can be confirmed by a G-banding method in the secondary selection. While the number of the chromosomes of the ES cell is preferably accounts for 100% of the number of the chromosomes in the normal cell, the ES cells are desirably cloned again to normal cells (for example, the number of the chromosomes is 2n=40 in mouse), when it is difficult to obtain the normal ES cells in relation to physical operations for establishing the cell line.

Although proliferating ability of the embryonic stem cell line obtained as described above is usually very good, sub-culture should be carefully performed since regenerative ability tends to be lost. For example, the cells are cultivated on appropriate feeder cells such as STO fibroblast cells in the presence of LIF (1 to 10,000 U/ml) in a carbon dioxide incubator (preferably about 5% of carbon dioxide and about 95% of air, or about 5% of carbon dioxide and about 90% of air) at 37° C. For sub-cultivation, the cells are divided into single cells by, for example, a trypsin treatment (usually about 0.001 to 0.5% trypsin/about 0.1 to 5 mM EDTA, preferably about 0.1% trypsin/about 1 mM EDTA), and seeded on the freshly prepared feeder cells. While such sub-cultivation is performed for every 1 to 3 days, the cells are observed at every sub-cultivation to abandon the cells having abnormal configurations.

The ES cells are able to differentiate into various cell types such as pariental cells, internal organ cells and heart muscle cells by monolayer cultivation up to high density, or by floating cultivation until cell clots are formed under an appropriate condition (M. J. Evans and M. H. Kaufman, Nature vol. 292, p154, 1981; G. R. Martin, Proceeding of national Academy of Science U.S.A, vol. 78, p7634, 1981; T. C. Doetshman et. al., Journal of Embryology and Experimental Morphology, vol. 87, p27, 1985). The DNA expression deficient cells of the invention obtained by differentiation of the ES cells of the invention is useful for in vitro cell biological investigations of the polypeptide of the invention.

The DNA expression deficient non-human mammal of the invention can be discriminated from normal animals by indirectly comparing the number of expression using the number of mRNAs of the animal by a conventional method.

The same non-human mammals as described above may be used.

The DNA of the invention can be knocked out in the DNA expression deficient non-human mammal of the invention by introducing the targeting vector prepared as described above into the mouse embryonic cells or mouse egg cells, and substituting the DNA of the invention on the chromosomes of the mouse embryonic stem cells or mouse egg cells with the DNA sequence in which the DNA of the invention is inactivated by introducing the targeting vector by homologous recombination of the gene.

The cells in which the DNA of the invention is knocked out can be judged by a Southern hybridization analysis using the DNA of the invention or the DNA sequence in the vicinity thereof as a probe, or by a PCR method using as primers the DNA sequence on the targeting vector and the DNA sequence in the neighbor region other than the DNA of the invention originating from the mouse used for the targeting vector. When the embryonic stem cells of the non-human mammal are used, the cell line in which the DNA of the invention is inactivated is cloned by homologous recombination of genes, the cells are injected into the embryos or blastocysts of the non-human mammal at eight cell stage, and chimera stem cells prepared are transplanted into the uterus of the non-human mammal in spurious pregnancy. The created animal is a chimera animal comprising the cells having loci of the normal DNA of the invention and loci of the artificially mutated DNA of the invention.

When the chimera animal has the DNA loci of the invention in which a part of germ cells has been mutated, individuals, in which all the tissues are artificially mutated, comprising the cells having the DNA loci of the invention are obtained by selecting from a population obtained by cross-breeding of the chimera animal with the normal animal by, for example, coat color judgment. The individuals obtained as described above are usually deficient in hetero-expression of the polypeptide of the invention, and the individuals deficient in hetero-expression of the polypeptide of the invention are cross-bred with each other to obtain individuals deficient in homo-expression of the polypeptide of the invention from babies therefrom.

When egg cells are used, a transgenic non-human mammal having the targeting vector introduced into the chromosome can be obtained by injecting the DNA solution into the nucleus of the egg cell by, for example, a microinjection method, and individuals mutated in the loci of the DNA of the invention by homologous recombination of genes are selected by comparing with the transgenic non-human mammal.

The individual in which the DNA of the invention is knocked out can be sub-bred in a usual breeding environment after confirming that the DNAs of the individual of the animal obtained by cross-breeding are also knocked out.

Acquirement and maintenance of the regenerative line may be carried out by a conventional method. Homozygote animals having the inactivated DNA in both homologous chromosomes can be obtained by cross-breeding of a male and female having the inactivated DNA. The homozygote animal can be efficiently obtained by breeding so that the mother animal gives rise to babies in a proportion of one normal individual and plural homozygotes. The homozygote and heterozygote having the inactivated DNA are sub-bred by cross-breeding of the heterozygote animals.

The non-human mammal in which the DNA of the invention is inactivated is quite useful for crating the non-human mammal in which the DNA of the invention is deficient.

Since the non-human mammal in which the DNA of the invention is inactivated is deficient in various biological activities induced from the polypeptide of the invention, the animal serves as a disease model caused by inactivation of the biological activity of the polypeptide of the invention. Accordingly, the mammal is useful for elucidation of the cause of these diseases and for therapy of these diseases.

(10a) Screening Method of Compounds Having Therapeutic and Preventive Effects Against Diseases Caused by Deletion and Damage of DNA of the Invention

The DNA expression deficient non-human mammal of the invention can be used for screening of compounds having therapeutic and preventive effects against the diseases (such as respiratory organ diseases, cancers, immune diseases, digestive tract diseases, circulatory organ diseases, internal secretion diseases, and bone and joint diseases) caused by deletion or damage of the DNA of the invention.

The invention provides a screening method of compounds or salts thereof having therapeutic and preventive effects against the diseases caused by deletion or damage of the DNA of the invention, wherein the test compounds are administered to the DNA expression deficient non-human mammals of the invention, and changes in the mammal are observed and measured.

Examples of the DNA expression deficient non-human mammals of the invention used in the screening method are the same as those described above.

Examples of the test compound include peptides, proteins, non-peptide compounds, synthetic compounds, fermentation products, cell extracts, plant extracts, animal tissue extracts and plasma. These compounds may be novel compounds, or compounds known in the art.

For example, the therapeutic and preventive effects of the test compound can be tested using changes of the animal in organs, tissues and diseases as indices after treating the DNA expression deficient non-human mammal of the invention with the test compound.

The therapeutic and preventive effects of the test compound can be also tested by quantifying the expression of the DNA (or mRNA) of the invention to compare with that in control groups after treating the animal as described above.

The methods for treating the test animal with the test compound include oral administration and intravenous injection, and the methods may be appropriately selected depending on the symptoms of the test animal and properties of the test compound. The dosage of the test compound may be also appropriately selected together with the administration method and the properties of the test compound.

The compound obtained by the screening method of the invention is selected from the test compounds described above. Since the compound has therapeutic and preventive effects against diseases (such as respiratory organ diseases, cancers, immune diseases, digestive tract diseases, circulatory organ diseases, internal secretion diseases, and bone and joint diseases) caused by deletion or damage of the polypeptide of the invention, the compound can be used as a safe and low toxic medicine for therapy and prevention against the diseases. The compounds derived from the compounds obtained by screening may be used as well.

The compounds obtained by the screening method above may form salts, which are salts with physiologically acceptable acids (such as inorganic and organic acids), and physiologically acceptable acid addition salts are preferable among them. Examples of the acids for forming the salts include inorganic acids (such as hydrochloric acid, phosphoric acid, hydrobromic acid and sulfuric acid) and organic acids (acetic acid, formic acid, propionic acid, fumaric acid, maleic acid, succinic acid, tartaric acid, citric acid, malic acid, oxalic acid, benzoic acid, methanesulfonic acid and benzenesulfonic acid).

The medicine containing the compound and the salt thereof obtained by the screening method can be produced by the same method as producing the medicine containing the polypeptide of the invention.

Since the formulation obtained as described above is safe and has low toxicity, it can be administered to mammals (such as human, rat, mouse, guinea pig, sheep, rabbit, swine, bovine, horse, cat, dog and monkey).

While the dosage of the compound and salts thereof differs depending on the disease, administration object and administration route, the dosage is about 0.1 to 100 mg, preferably about 1.0 to 50 mg, and more preferably about 1.0 to 20 mg a day for an adult (body weight 60 kg) when the compound is orally administered for therapy of the respiratory organ diseases. While the dosage of non-oral administration also differs depending on the administration object and disease, the dosage is about 0.01 to 30 mg, preferably about 0.1 to 20 mg, and more preferably about 0.1 to 10 mg a day for an adult (body weight 60 kg) when the compound is administered by intravenous injection as an injection agent for therapy of the respiratory organ diseases. A dosage converted into a body weight of 60 kg may be administered for other mammals.

(10b) Screening Method of Compounds that Promote or Inhibit the Activity of Promoter Against DNA of the Invention

The invention provides a method for screening a compound or a salt thereof that promotes or inhibits the activity of the promoter against the DNA of the invention, wherein the test compound is administered to the DNA expression deficient non-human mammal of the invention to detect expression of the reporter gene.

In the screening method above, the DNA of the invention is inactivated in the DNA expression deficient non-human mammal of the invention, and the reporter gene is expressed under the control of the promoter against the DNA of the invention.

Examples of the test compound are the same as those described above.

The reporter genes are also the same as described above, and β-galactosidase gene (lacZ), soluble alkali phosphatase gene or luciferase gene is favorably used.

Since the reporter gene is under the control of the promoter against the DNA of the invention in the DNA expression deficient mammal of the invention in which the DNA of the invention is substituted with the reporter gene, the activity of the promoter can be detected by tracing expression of a substance encoded by the reporter gene.

For example, when a part of the DNA region encoding the polypeptide of the invention is substituted with β-galactosidase gene (lacZ) derived from Escherichia coli, β-galactosidase is expressed in place of the polypeptide of the invention in the tissue where the polypeptide of the invention has been intrinsically expressed. Accordingly, expression of β-galactosidase in an animal can be readily observed by staining the tissue using a substrate of β-galactosidase such a 5-bromo-4-chloro-3-indolyl-α-galactopyranoside (X-gal). For example, a mouse deficient in the polypeptide of the invention, or a slice of the tissue thereof is fixed with glutaraldehyde, the sample is stained with a staining solution containing X-gal at room temperature or at 37° C. for 30 minutes to 1 hour after washing with a phosphate buffer saline (PBS), the tissue sample is washed with 1 mM EDTA/PBS to stop the galactosidase reaction, and the stained sample is observed. The mRNA encoding lacZ may be detected by a conventional method.

The compound or the salt thereof obtained by the screening method above is selected from the test compounds above, and the compound promotes or inhibits the activity of the promoter against the DNA of the invention.

The compounds obtained by the screening method above may form salts, and salts with physiologically acceptable (such as inorganic acids) and bases (such as organic acids) are used. Physiologically acceptable acid addition salts are particularly preferable. Examples of the acids for forming the salts include inorganic acids (such as hydrochloric acid, phosphoric acid, hydrobromic acid and sulfuric acid), and organic acids (such as acetic acid, formic acid, propionic acid, fumaric acid, maleic acid, succinic acid, tartaric acid, citric acid, malic acid, oxalic acid, benzoic acid, methanesulfonic acid and benzenesulfonic acid)s.

Since the compound for promoting the activity of the promoter against the DNA of the invention can promote expression of the polypeptide of the invention to promote the function of the polypeptide, it is useful as a safe therapeutic and preventive agent having low toxicity against the diseases such as respiratory organ diseases, cancers, immune diseases, digestive tract diseases, circulatory organ diseases, internal secretion diseases, and bone and joint diseases.

The compounds induced from the compounds obtained by the screening method above are also used as described above.

The medicine containing the compound and the salt thereof obtained by the screening method above can be produced by the same method as producing the medicine containing the polypeptide or the salt thereof of the invention.

Since the formulation obtained as described above is safe and has low toxicity, it can be administered to rat, human, mouse, guinea pig, rabbit, sheep, swine, bovine, horse, cat, dog and monkey.

While the dosage of the compound and salts thereof differs depending on the disease, administration object and administration route, the dosage is about 0.1 to 100 mg, preferably about 1.0 to 50 mg, and more preferably about 1.0 to 20 mg a day for an adult (body weight 60 kg) when the compound for promoting the activity of the promoter against the DNA of the invention is orally administered for therapy of the respiratory organ diseases. While the dosage of non-oral administration also differs depending on the administration object and disease, the dosage is about 0.01 to 30 mg, preferably about 0.1 to 20 mg, and more preferably about 0.1 to 10 mg a day for an adult (body weight 60 kg) when the compound is administered by intravenous injection as an injection agent for therapy of the respiratory organ diseases. A dosage converted into a body weight of 60 kg may be administered for other mammals.

When the compound for inhibiting the activity of the promoter against the DNA of the invention is orally administered for therapy of the respiratory organ diseases, the dosage is about 0.1 to 100 mg, preferably about 1.0 to 50 mg, and more preferably about 1.0 to 20 mg a day for an adult (body weight 60 kg). While the dosage of non-oral administration differs depending on the administration object and disease, the dosage is about 0.01 to 30 mg, preferably about 0.1 to 20 mg, and more preferably about 0.1 to 10 mg a day for an adult (body weight 60 kg) when the compound is administered by intravenous injection as an injection agent for therapy of the respiratory organ diseases. A dosage converted into a body weight of 60 kg may be administered for other mammals.

The DNA expression deficient non-human mammal of the invention is quite useful for screening the compound for promoting or inhibiting the activity of the promoter against the DNA of the invention, and is able to largely contribute to elucidation of causes of various diseases ascribed to deficiency of expression of the DNA of the invention and to the development of the therapeutic and preventive agent.

Since the gene encoding the polypeptide of the invention is specifically expressed in the trachea and fetus lung, the promoter sequence of the gene is favorable as a promoter for allowing a desired protein (such as an any useful gene product) to be specifically expressed in non-human homotherms. Examples of the non-human homotherm include those described as the homotherm above.

The present invention provides a method for allowing the desired protein (such as an any useful gene product) to be preferentially expressed in the trachea and lung of the non-human homotherms, wherein the a sequence encoding the desired protein (such as an any useful gene product) is linked at the downstream (3′-terminal side) of the promoter region of the gene encoding the polypeptide containing an amino acid sequence represented by SEQ ID NO: 2 or SEQ ID NO: 18, and the sequence is introduced into the non-human animal.

Examples of the desired protein (such as an any useful gene product) include cytokines (for example interleukin, interferon, chemokine and hematopoietic factor), proliferation factor (for example EFG (epidermal growth factor) or substances having substantially the same activity thereof such as EGF and haleglin (HER 2 ligand)), insulin or substances having substantially the same activity thereof (for example insulin, IGF-1 (insulin-like growth factor-1) and IGF-2), FGF (fibroblast growth factor) or substances having substantially the same activity thereof (for example aFGF, bFGF, KFG (keratinocyte growth factor), HGF (hepatocyte growth factor) and FGF-10), other growth factors (for example CSF (colony stimulating factor), EPO (eryyjropoietin), IL-2 (interleukin-2), NGF (nerve growth factor), PDFG (platelet-derived growth factor) and TGFβ (transforming growth factor)), hormones (for example luteinizing hormone release hormone (LH-RH), growth hormone, growth hormone release hormone (GH-RH), prolactin, melanocyte stimulating hormone, thyroid hormone release hormone, thyroid stimulating hormone, luteinizing hormone forming hormone, luteinizing hormone, follicle stimulating hormone, gastrin, motilin, somatostatin, secretin, glucagon, PACAP and VIP), digestive enzymes (for example amylase, pepsinogen and lipase), antibodies against pathogens (for example antigens against pathogenic bacteria such as pathogenic Salmonella, antibodies against pathogenic viruses such as influenza virus, and antibodies against parasites such as Echinococcus), and antibacterial polypeptides (for example cecropin, histatin, indolysidine, proteglin, difensin and lysozyne).

The desired protein above is able to:

[1] Enhance and regulate immunological activity of the non-human homotherm by allowing cytokines to be specifically expressed in the trachea and lung; and

[2] Enhance resistance of the non-human homotherms against infectious diseases by allowing antibacterial polypeptides to be specifically expressed in the trachea and lung.

The methods, by which DNAs encoding desired proteins (any useful gene products) are linked at the downstream (3′-terminal side) of the promoter region of the gene encoding the polypeptide comprising the amino acid sequence represented by SEQ ID NO: 2 or SEQ ID NO: 18, and the desired protein (any useful gene products) is allowed to be specifically expressed at the trachea and lung of the non-human homotherms by introducing it into the non-human homotherms, will be described in detail hereinafter.

The promoter of the gene encoding the polypeptide comprising the amino acid sequence represented by SEQ ID NO: 2 or SEQ ID NO: 18 is obtained by the methods known in the art such as colony hybridization, plaque hybridization and PCR (for example, methods described in Molecular Cloning, 2nd edition, J. Sambrook et. al., Cold Spring Harbor Lab. Press, 1989). The region having the promoter activity can be identified by the methods known in the art such as a reporter assay (for example the method described in Analytical Biochemistry, vol. 188, p245, 1990).

In order to ligate the desired protein (any useful gene products) downstream of the promoter obtained as described above (at 3′-terminal side), the aim can be accomplished by a publicly known method for constructing a plasmid using T4 DNA ligase (for example, the method described in Molecular Cloning, 2nd edition, J. Sambrook et. al., Cold Spring Harbor Lab. Press, 1989).

For introducing the DNA ligated with the DNA encoding the desired protein (any useful gene products) downstream of the promoter (at 3′-terminal side) into the non-human warm-blooded animal, the methods using electroporation, gene gun or retrovirus vector (for example the method described in Blood Cells, vol. 17, p407, 1991), and by the method using adenovirus vector (for example the method described in Pathology, vol. 30, p335, 1998) can be used.

When the bases and amino acids are represented by abbreviations in the specification and drawings of the invention, they are based on the abbreviations by IUPAC-IUB Commission on Biological Nomenclature or customary used abbreviations in the field of art. Examples of them are described below. When the amino acid comprises optical isomers, the abbreviation denotes a L-form unless otherwise noticed.

| | |
|---|---|
| DNA | deoxyribonucleic acid |
| cDNA | complementary deoxyribonucleic acid |
| A | adenine |
| T | thymine |
| G | guanine |
| C | cytosine |
| RNA | ribonucleic acid |
| mRNA | messenger ribonucleic acid |
| dATP | deoxyriboadenosine triphosphate |
| dTTP | deoxythymidine triphosphate |
| dGTP | deoxyguanosine triphosphate |
| dCTP | deoxycytidine triphosphate |
| ATP | adenosine triphosphate |
| EDTA | ethylenediamine tetraacetic acid |
| SDS | sodium dodecyl sulfate |
| Gly | glycine |
| Ala | alanine |
| Val | valine |
| Leu | leucine |
| Ile | isoleucine |
| Ser | serine |
| Thr | threonine |
| Cys | cysteine |
| Met | methionine |
| Glu | glutamic acid |
| Asp | aspartic acid |
| Lys | lysine |
| Arg | arginine |
| His | histidine |
| Phe | phenylalanine |
| Tyr | tyrosine |
| Trp | tryptophane |
| Pro | proline |
| Asn | asparagine |
| Gln | glutamine |
| pGlu | pyrroglutamic acid |

The substituents, protective groups and reagents used in the specification are denoted by the symbols below:

| | |
|---|---|
| Me | methyl groups |
| Et | ethyl group |
| Bu | butyl group |
| Ph | phenyl group |
| TC | thyazolidine-4(R)-carboxamide group |
| Tos | p-toluenesulfonyl |
| CHO | formyl |
| Bzl | benzyl |
| Cl₂-Bzl | 2,6-dichlorobenzyl |
| Bom | benzyloxymethyl |
| Z | benzyloxycarbonyl |
| Cl-Z | 2-chlorobenzyloxycarbonyl |
| Br-Z | 2-bromobenzyloxycarbonyl |
| Boc | t-butoxycarbonyl |
| DNP | dinitrophenyl |
| Trt | trityl |
| Bum | t-butoxymethyl |
| Fmoc | N-9-fluorenylmethoxycarbonyl |
| HOBt | 1-hydroxybenztriazole |
| HOOBt | 3,4-dihydro-3-hydroxy-4-oxo-1,2,3-benzotriazine |
| HONB | 1-hydroxy-5-norbornen-2,3-dicarboxyimide |
| DCC | N,N′-dicyclohexylcarbodiimide |

The SEQ ID NO: in the sequence listing of the specification of the invention is as follows:

[SEQ ID NO: 1]

This shows the base sequence of the novel human cDNA fragment obtained in Example 1;

[SEQ ID NO: 2]

This shows the amino acid sequence of human precursor polypeptide;

[SEQ ID NO: 3]

This shows the base sequence of DNA encoding the human precursor polypeptide;

[SEQ ID NO: 4]

This shows the amino acid sequence of human polypeptide of the invention;

[SEQ ID NO: 5]

This shows the base sequence of DNA encoding the human polypeptide of the invention;

[SEQ ID NO: 6]

This shows the amino acid sequence of human mature peptide 1;

[SEQ ID NO: 7]

This shows the base sequence of DNA encoding the human mature peptide 1;

[SEQ ID NO: 8]

This shows the amino acid sequence of human mature peptide 2;

[SEQ ID NO: 9]

This shows the base sequence of DNA encoding the human mature peptide 2;

[SEQ ID NO: 10]

This shows the base sequence of primer (sense strand) used in Example 1;

[SEQ ID NO: 11]

This shows the base sequence of primer (antisense strand) used in Example 1;

[SEQ ID NO: 12]

This shows the base sequence of primer (sense strand) used in Example 2;

[SEQ ID NO: 13]

This shows the base sequence of primer (antisense strand) used in Example 2;

[SEQ ID NO: 14]

This shows the base sequence of primer (sense strand) used in Example 3;

[SEQ ID NO: 15]

This shows the base sequence of primer (antisense strand) used in Example 3;

[SEQ ID NO: 16]

This shows the amino acid sequence of polypeptide encoded by the expression vector constructed in Example 3;

[SEQ ID NO: 17]

This shows the base sequence encoding the polypeptide encoded by the expression vector constructed in Example 3;

[SEQ ID NO: 18]

This shows the amino acid sequence of mouse precursor polypeptide;

[SEQ ID NO: 19]

This shows the base sequence of DNA encoding the mouse precursor polypeptide;

[SEQ ID NO: 20]

This shows the amino acid sequence of the mouse polypeptide of the invention;

[SEQ ID NO: 21]

This shows the base sequence of DNA encoding the mouse polypeptide of the invention;

[SEQ ID NO: 22]

This shows the amino acid sequence of mouse mature peptide 1;

[SEQ ID NO: 23]

This shows the base sequence of DNA encoding the mouse mature peptide 1;

[SEQ ID NO: 24]

This shows the amino acid sequence of mouse mature peptide 2; and

[SEQ ID NO: 25]

This shows the base sequence of DNA encoding the mouse mature peptide 2.

The transformed Escherichia coli XL10-Gold/pDRL138 h obtained in Example 1 below was deposited with Institute for Fermentation, Osaka (IFO), located at 2-17-85 Juso-Honmachi, Yodogawa-ku, Osaka 532-8686, as a deposit number IFO 16511 since Dec. 6, 2000, and was deposited with National Institute of Advanced Industrial Science and Technology, International Patent Organism Depository (formerly National Institute of Bioscience and Human Technology (NIBH), Agency of Industrial Science and Technology, Ministry of Trade and Industry), located at Chuo-6, 1-1-1 Higashi, Tukuba-shi, Ibaraki 305-8566, as a deposit number FERM BP-7399 since Dec. 19, 2000.

### EXAMPLES

While the invention is described in detail with reference to examples, the invention is restricted to these examples. Gene manipulation using Escherichia coli was carried out according to the method described in Molecular Cloning.

### Example 1

### Cloning of cDNA encoding human DRL138

As a result of retrieval of a sequence comprising both a signal sequence for secretion and basic amino acid pairs (such as Arg-Arg and Lys-Arg) in the same open reading frame (ORF) in the human gene database, a novel sequence derived from chromosome 10 was found in the human gene sequence. In order to confirm whether mRNAs containing this gene sequence are actually transcribed, and whether the predicted ODF is correct, cDNAs were obtained by the following method.

Into 3 μl each of the human ovary derived cDNA (Multiple Tissue cDNA Panel,

| | |
|---|---|
| 10 pmol of oligo DNA | |
| CCTGGACGGAGCCCCTACCT, | (SEQ ID NO: 10) |
| 10 pmol of oligo DNA | |
| GATTGCTGGAACACGGGGATGC. | (SEQ ID NO: 11) |

10 μl of distilled water and 20 μl of Premix Taq (Ex Version, Takara) were added to a final volume of 40 μl. The mixture was subjected to 35 cycles of reaction (94° C., 30 seconds and 68° C., 2 minutes) using a thermal cycler (GeneAmp PCR System Model 9700, Perkin-Elmer) after incubating at 96° C. for 1 minute, to get about 0.5 kb of amplified PCR fragment. Then, the DNA fragment was purified by agarose gel electrophoresis, and cloned using pCR2.1-TOPO (Invitrogen) for base sequence determination. The plasmid was introduced into Escherichia coli XL10-Gold competent cells (Stratagene). The clone harboring the plasmid in which the 0.5 kb DNA fragment has been introduced was selected from the colonies of ampicillin resistant transformant appearing on a LB agar medium containing ampicillin, therefrom preparing the plasmid DNA (pDRL138 h). Subsequently, pDRL138 h was sequenced using two primer DNAs (PRM-007 and PRM-008, TOYOBO) for sequence primers and ABI PRISM™ BigDye Terminator Cycle Sequencing FS Ready Reaction Kit (Perkin-Elmer), and the sequence was decoded with a DNA sequencer, ABI PRISM™ 377 (Perkin-Elmer).

As a result, it was revealed that a novel cDNA fragment consisting of 513 bases represented by SEQ ID NO: 1, which has no homology at all with the publicly known base sequences, was inserted into pDRL138 h. The cDNA contained the base sequence corresponding to the open reading frame represented by SEQ ID NO: 3, which encodes the novel protein (referred to as DRL138 polypeptide or DRL138 hereinafter) consisting of 138 amino acid residues represented by SEQ ID NO: 2. It was confirmed from the result above that the gene had been transcribed and translated. It was also made clear that the gene is cDNA of the novel polypeptide containing signal sequences from the fact that ORF encoding the polypeptide having no homology at all with the publicly known amino acid sequences had been confirmed.

### Example 2

### Analysis of Expression Site in Human

Membrane filters on which poly(A)⁺ RNA of each human tissue had been previously blotted (Human 12-Lane MTN Blot (catalogue No. 7780-1, Clontech), Human 12-Lane MTN Blot II (catalogue No. 7784-1, Clontech), Northern Lights Human Multiple mRNA Blot IV (catalogue No. 11396-017, Gibco), Hu Fetal mRNA Blot I (catalogue No. D2801-08, Invitrogen), and Hu Fetal mRNA Blot II (catalogue No. D2808-08, Invitrogen)), and a membrane filters on which poly(A)+ RNA or DNA of each human tissue had been previously dot-blotted (Human Multiple Tissue Expression (MTE) Array (catalogue No. 7775-1, Clontech)) were subjected to pre-hybridization at 60° C. in a hybridization buffer (0.5M sodium phosphate buffer (pH 7.2) containing 1 mM EDTA and 7% SDS). On the other hand, a 0.4 kb DNA fragment obtained by amplification by PCR using cDNA derived from the human ovary (Multiple Tissue cDNA Panel, Clontech K1412-1) as a template, an oligo DNA represented by SEQ ID NO: 12 (5′-AGGCAGMGCTTCGGGTTGATGA-3′) as a sense strand primer, and an oligo DNA represented by SEQ ID NO: 13 (5′AGGGGMGCCACTGTAGGTTTG-3′) as an antisense strand primer was labeled for use as a probe using [α-³²P]dCTP (catalogue No. NEG-513Z, DuPont) and a random primer labeling kit (catalogue No. RPN1601Y, Amersham Bioscience). After pre-hybridizing in the hybridization buffer (0.5 M sodium phosphate buffer (pH 7.2) containing 1 mM of EDTA and 7% of SDS) at 60° C. for 30 minutes, the hybridization was performed in the hybridization buffer (0.5 M sodium phosphate buffer (pH 7.2) containing 1 mM of EDTA and 7% of SDS) containing the labeled probe at 60° C. for 18 hours. After finally washing the filters with 0.1×SSC (20×SSC=3 M sodium chloride, 0.3 M sodium citrate) and 0.1% SDS at 50° C. or 60° C., bands or spots hybridized with the probe was detected by autoradiogram. The BAS-2000 (Fuji Film) was used for detection. As a result, it was found that the size of the mRNA encoding the polypeptide of the invention is about 0.9 kb, and the RNA is able to hybridize with poly(A)⁺ RNA derived from the trachea and fetus lung.

### Example 3

### Construction of the Expression Vector of Human DRL138

The DNA fragment encoding human DRL 138 was obtained by the following PCR method. That is, 50 μl of the mixture containing 20 pmol each of an oligo DNA represented by SEQ ID NO: 14 (5′-CTACGAATTCCACCATGACTTGGAGACAGGCCGTCC-TGCT-3′) as a sense strand primer and an oligo DNA represented by SEQ ID NO: 15 (5′-TTCAGGTCGACGGTGTGGTGG-CGGTTGTAGAGATAG-3′) as an antisense strand primer, 5 11 of 10×Advantage® 2 PCR Buffer (Clontech), 1 μl of 50×dNTP mix (Clontech), 1 μl of 50×Advantage 2 Polymerase Mix (Clontech) and 1 ng of the plasmid pDRL 138 h as a template DNA described in Example 1 was prepared. The mixture was subjected to PCR reactions comprising 96° C./1 minute followed by 15 cycles set to 96° C./5 seconds and 68° C./30 seconds, and finally 68° C./2 minutes using Thermal Cycler (GeneAmp (registered trademark) PCR system model 9700, Applied Biosystems). After double digestion with restriction enzymes EcoRI and SalI of the solution after completing the reaction, unreacted primers and short DNA fragments formed by restriction enzyme digestion were removed using QIAQuick Gel PCR Purification Kit (Qiagen). The fragment obtained was subcloned to pCAN618.FLAG vector, and the plasmid was introduced into Escherichia coli XL10-Gold (Stratagene) competent cells. The clones retaining the plasmid in which exogenous DNA fragments are inserted were selected from the colonies of ampicillin resistant transformant appearing on the LB agar culture medium containing ampicillin, and the plasmid pDRL138h-FLAG was prepared.

As shown in SEQ ID NO: 16, pDRL138h-FLAG contains a DNA fragment consisting of 441 base pairs represented by SEQ ID NO: 17 encoding a human DRL138h-FLAG fusion protein in which a Val residue and subsequently a FLAG sequence, Asp-Tyr-Lys-Asp-Asp-Asp-Asp-Lys are presented at the C-terminal of the DRL polypeptide. The FLAG sequence serves as an epitope (antigen recognition site) for detecting the gene products.

### Example 4

### Expression in COS-7 Cells

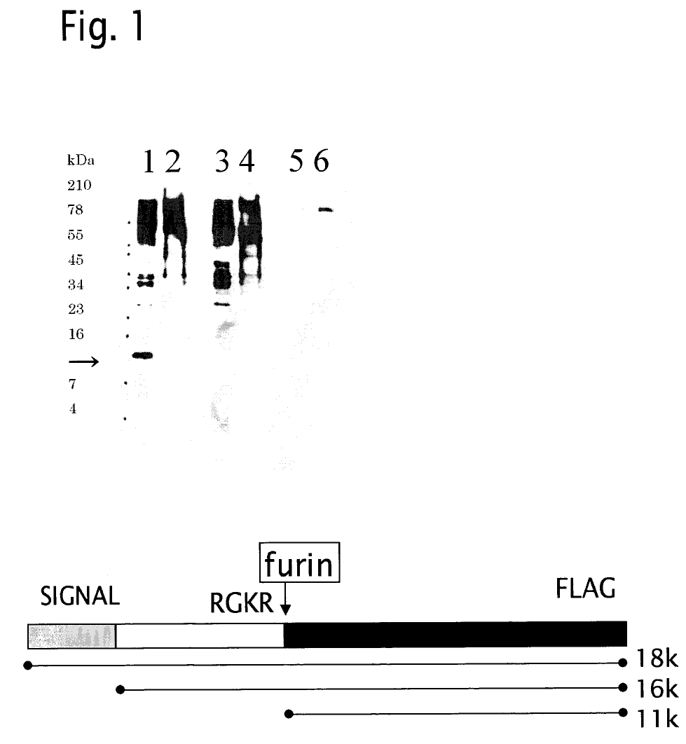

In order to confirm that the human DRL protein is a secretion protein, using COS-7 cells, it was verified whether the DRL138h-FLAG fusion protein was expressed to secrete into the culture medium. COS-7 cells were seeded on a six-well plate so that the number of the cells is 2×10⁵ cells/well a day before transfection of the expression vector, and the cells were cultivated in a CO₂ incubator for 24 hours at 37° C. in a DMEM culture medium (Gibco) containing 10% FBS (Japan Racing Horse Association). After transfection using 1 μg/well of the expression plasmid pDRL138h-FLAG constructed in Example 3 and Effectene (Qiagen), the culture medium was changed to a serum-free DMEM culture medium containing 1 ml of 0.1 mM pABSF (Wako Pure Chemical Industries) and 0.5% CHAPS (Dojindo Laboratories) 24 hours after transfection, followed by additional cultivation for 48 hours. The supernatant of the culture medium was centrifuged after transferring it in Eppendorf sample tubes, and the supernatant free from the cells was concentrated to about 1/20 using Centicon TM-3 ultrafiltration membrane (Millipore). The same volume of Tris-Tricine SDS-PAGE sample buffer (Tefco) was added to the concentrated supernatant of the culture medium. After washing the cells with 1 ml of PBS (Gibco) twice, 500 μl of Tris-Tricine SDS-PAGE sample buffer was added to the cells. After heat-treating these samples at 95° C. for 5 minutes, the samples were subjected to electrophoresis on 16% Peptide-PAGE mini (Tefco), and the samples were transferred from the gel onto Hybond P-membrane (Amersham Bioscience). Subsequently, after blocking the Hybond P-membrane with PBS containing 0.1% Tween-20 and 50% Block Ace (Snow Brand Milk Products), the DRL138h-FLAG fusion protein was detected by Western blotting. The protein was detected by a reaction using an anti-FLAG M2 mouse IgG monoclonal antibody (1/2000 dilution, Sigma) as a primary antibody and HRP labeled anti-mouse IgG sheep antibody (1/2000 dilution, Amersham) as a secondary antibody. The protein was detected by chemiluminescence using ECL Western blotting kit (Amersham) for light emission and Hyper-film ECL for detection. It was revealed from the results that the human DRL138h-FLAG fusion protein was secreted in the supernatant of the cell culture medium, and the molecular weight thereof was about 11 kDa. No protein was detected in the cell fraction. The DRL138h-FLAG fusion protein consists of 147 amino acid residues, and the calculated molecular weight thereof was 17,656. On the other hand, a sequence (Arg-Gly-Lys-Arg) that is recognizable as a substrate by a protein processing enzyme (furin) is present at the site from 61 through 64 of the DRL138h-FLAG fusion protein. The calculated molecular weight from Ser 65 to the C-terminal is 10,531, which is nearly identical to the band size observed in Western blotting. From these results, it is considered that the human DRL138 protein is subjected to restricted degradation at the cleavage site with a sequence of (Arg-GlyLys-Arg↓) with the protein processing enzyme (furin), and is secreted out of the cell [FIG. 1].

### Example 5

### Sequence of Mouse DRL138

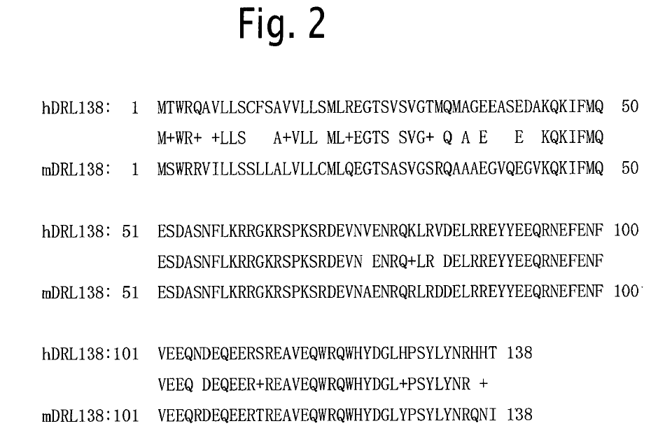

The sequence of mouse DRL138 was estimated as follows by retrieving the mouse gene information database. The mouse gene information database was retrieved homology searching (BLAST searching) using the base sequence of human DRL138 represented by SEQ ID NO: 1 and amino acid sequence represented by SEQ ID NO: 2 as query sequences, respectively. As a result, the amino acid sequence of the polypeptide of mouse DRL138 was conjectured to be represented by SEQ ID NO: 18. The base sequence encoding the amino acid sequence of mouse DRL138 is considered to be SEQ ID NO: 19. When the amino acid sequences of human DRL138 and mouse DRL138 were aligned in the order from the N-terminals, 109 amino acid residues of the 138 amino acid residues were consistent with each other, showing that the amino acid sequences are conserved quite well in both proteins [FIG. 2].

### INDUSTRIAL APPLICABILITY

The polypeptide of the invention, and the DNA coding the polypeptide and antisense DNA thereof can be used as the diagnostic, therapeutic and preventive agents for diseases such as respiratory diseases, cancers, immune diseases, infectious diseases, digestive tract diseases, circulatory diseases, endocrinopathy, and bone and joint diseases. The polypeptide of the invention is also useful as reagents for screening the compound or salts thereof for promoting or inhibiting the activity of the polypeptide of the invention. Furthermore, since the antibody against the polypeptide of the invention can specifically recognize the polypeptide of the invention, the antigen can be used for detection, quantification and neutralization of the polypeptide of the invention in a testsolution.

1. A polypeptide, or an amide, an ester or a salt thereof comprising the same or substantially the same amino acid sequence as the amino acid sequence represented by SEQ ID NO: 4 (human full length sequence with no signals).

2. The polypeptide, or an amide, an ester or a salt thereof according to claim 1, comprising an amino acid sequence represented by SEQ ID NO: 4 (human full length sequence with no signals), SEQ ID NO: 2 (human full length sequence containing signals), SEQ ID NO: 16 (human full length sequence+FLAG sequence), SEQ ID NO: 18 (mouse full length sequence containing signals), or SEQ ID NO: 20 (mouse full length sequence with no signals).

3. A polypeptide, or an amide, an ester or a salt thereof comprising the same or substantially the same amino acid sequence as the amino acid sequence represented by SEQ ID NO: 6 (human mature sequence 1), SEQ ID NO: 8 (human mature sequence 2), SEQ ID NO: 22 (mouse mature sequence 1), or SEQ ID NO: 24 (mouse mature sequence 2).

4. The polypeptide, or an amide, an ester or a salt thereof according to claim 3, comprising an amino acid sequence represented by SEQ ID NO: 6, SEQ ID NO: 8, SEQ ID NO: 22 or SEQ ID NO: 24.

5. The polypeptide according to claim 1, or a partial peptide, an amide, an ester or a salt of the peptide according to claim 3.

6. A polynucleotide comprising a polynucleotide encoding the polypeptide according to claim 1.

7. The polynucleotide according to claim 6, which is a DNA.

8. The DNA according to claim 7, having a base sequence represented by SEQ ID NO: 1, SEQ ID NO: 3, SEQ ID NO: 5, SEQ ID NO: 17, SEQ ID NO: 19 or SEQ ID NO: 21.

9. A polynucleotide comprising a polynucleotide encoding the peptide according to claim 3.

10. The polynucleotide according to claim 9, which is a DNA.

11. The DNA according to claim 10, having a base sequence represented by SEQ ID NO: 7, SEQ ID NO: 9, SEQ ID NO: 23 or SEQ ID NO: 25.

12. A recombinant vector comprising the polynucleotide according to claim 6 or 9.

13. A transformant transformed by the recombinant vector according to claim 12.

14. A method for producing the polypeptide, or the amide, ester or salt thereof according to claim 1, or the polypeptide, or the amide, ester or salt thereof according to claim 3, comprising the step of cultivating the transformant according to claim 13 to produce and accumulate the polypeptide according to claim 1 or the peptide according to claim 3.

15. An antibody against the polypeptide, or the amide, ester or salt thereof according to claim 1, the peptide, or the amide, ester or salt thereof according to claim 3, or the partial peptide, or the amide, ester or salt thereof according to claim 5.

16. A screening method of a compound or a salt thereof for promoting or inhibiting the activity of the polypeptide, or the amide, ester or salt thereof according to claim 1, the peptide, or the amide, ester or salt thereof according to claim 3, or the partial peptide, or the amide, ester or salt thereof according to claim 5, which comprises using the polypeptide, or the amide, ester or salt thereof according to claim 1, the peptide, or the amide, ester or salt thereof according to claim 3, or the partial peptide, or the amide, ester or salt thereof according to claim 5.

17. A screening kit of the compound or the salt thereof for promoting or inhibiting the activity of the polypeptide, or the amide, ester or salt thereof according to claim 1, the peptide, or the amide, ester or salt thereof according to claim 3, or the partial peptide, or the amide, ester or salt thereof according to claim 5 comprising the polypeptide, or the amide, ester or salt thereof according to claim 1, the peptide, or the amide, ester or salt thereof according to claim 3, or the partial peptide, or the amide, ester or salt thereof according to claim 5.

18. The compound or the salt thereof for promoting or inhibiting the activity of the polypeptide, or the amide, ester or salt thereof according to claim 1, the peptide, or the amide, ester or salt thereof according to claim 3, or the partial peptide, or the amide, ester or salt thereof according to claim 5, which is obtainable using the screening method according to claim 16 or the screening kit according to claim 17.

19. A medicine comprising the compound or the salt thereof for promoting or inhibiting the activity of the amide, ester or salt thereof according to claim 1, the peptide, or the amide, ester or salt thereof according to claim 3, or the partial peptide, or the amide, ester or salt thereof according to claim 5, which is obtainable using the screening method according to claim 16 or screening kit according to claim 17.

20. A preventive or therapeutic agent of respiratory diseases, cancers, immune diseases, infectious diseases, digestive tract diseases, circulatory diseases, bone and joint diseases or endocrinopathy comprising the compound or the salt thereof for promoting the activity of the amide, ester or salt thereof according to claim 1, the peptide, or the amide, ester or salt thereof according to claim 3, or the partial peptide, or the amide, ester or salt thereof according to claim 5, which is obtainable using the screening method according to claim 16, or the screening kit according to claim 17.

21. A medicine comprising the polypeptide, or the amide, ester or salt thereof according to claim 1, or the peptide, or the amide, ester or salt thereof according to claim 3.

22. A preventive or therapeutic agent of respiratory diseases, cancers, immune diseases, infectious diseases, digestive tract diseases, circulatory diseases, bone and joint diseases or endocrinopathy comprising the polypeptide, or the amide, ester or salt thereof according to claim 1, or the peptide, or the amide, ester or salt thereof according to claim 3.

23. A medicine comprising the polynucleotide according to claim 6 or 9.

24. The medicine according to claim 23, which is the preventive or therapeutic agent of respiratory diseases, cancers, immune diseases, infectious diseases, digestive tract diseases, circulatory diseases, bone and joint diseases or endocrinopathy.

25. A diagnostic agent comprising the antibody according to claim 15.

26. A medicine comprising the antibody according to claim 15.

27. An antisense DNA having a base sequence or a part thereof complementary or substantially complementary to the DNA encoding the polypeptide according to claim 1 or the peptide according to claim 3, and having a function for suppressing the expression of the DNA.

28. A medicine comprising the antisense DNA according to claim 27.

29. A gene diagnosis agent comprising the polynucleotide according to claims 6 and 9, or the antisense DNA according to claim 27.

30. A preventive or therapeutic method of respiratory diseases, cancers, immune diseases, infectious diseases, digestive tract diseases, circulatory diseases, bone and joint diseases or endocrinopathy comprising administering to mammals, an effective dose of the polypeptide, or the amide, ester or salt thereof according to claim 1, or the peptide, or the amide, ester or salt thereof according to claim 3.

31. The preventive or therapeutic method of respiratory diseases, cancers, immune diseases, infectious diseases, digestive tract diseases, circulatory diseases, bone and joint diseases or endocrinopathy comprising administering an effective dose of the polynucleotide according to claim 6 or 9 to mammals.

32. The preventive or therapeutic method of respiratory diseases, cancers, immune diseases, infectious diseases, digestive tract diseases, circulatory diseases, bone and joint diseases or endocrinopathy comprising administering, to mammals, an effective dose of the compound or the salt thereof for promoting or inhibiting the activity of the polypeptide, or the amide, ester or salt thereof according to claim 1, the peptide, or the amide, ester or salt thereof according to claim 3, or the partial peptide, or the amide, ester or salt thereof according to claim 5, which is obtainable using the screening method according to claim 16 or the screening kit according to claim 17.

33. Use of the polypeptide, or the amide, ester or salt thereof according to claim 1, or the peptide, or the amide, ester or salt thereof according to claim 3 for producing the preventive or therapeutic agent of respiratory diseases, cancers, immune diseases, infectious diseases, digestive tract diseases, circulatory diseases, bone and joint diseases or endocrinopathy.

34. Use of the polynucleotide according to claim 6 or 9 for producing the preventive or therapeutic agent of respiratory diseases, cancers, immune diseases, infectious diseases, digestive tract diseases, circulatory diseases, bone and joint diseases or endocrinopathy.

35. Use of the compound or the salt thereof for promoting the activity of the polypeptide, or the amide, ester or salt thereof according to claim 1, the peptide, or the amide, ester or salt thereof according to claim 3, or the partial peptide, or the amide, ester or salt thereof according to claim 5, which is obtainable using the screening method according to claim 16 or screening kit according to claim 17 for producing the preventive or therapeutic agent of respiratory diseases, cancers, immune diseases, infectious diseases, digestive tract diseases, circulatory diseases, bone and joint diseases or endocrinopathy.

36. A transgenic non-human mammal having the exogenous DNA according to claim 7 or 10 or mutated DNA thereof.

37. The non-human mammal according to claim 36, wherein the non-human mammal is a rodent.

38. The non-human mammal according to claim 37, wherein the rodent is a mouse.

39. A recombinant vector comprising the exogenous DNA or the mutated DNA thereof according to claim 7 or 10, which is capable of being expressed in the non-human mammal.

40. An embryonic stem cell of the non-human mammal, in which the DNA according to claim 7 or 10 is inactivated.

41. The embryonic stem cell according to claim 40, wherein the DNA is inactivated by introducing a reporter gene.

42. The embryonic stem cell according to claim 40, which is resistant to neomycin.

43. The embryonic stem cell according to claim 40, wherein the non-human mammal is a rodent.

44. The embryonic stem cell according to claim 43, wherein the rodent is a mouse.

45. A non-human mammal deficient in DNA expression, in which the DNA according to claim 7 or 10 is inactivated.

46. The non-human mammal according to claim 45, wherein the DNA is inactivated by introducing the reporter gene, which is able to be expressed under the control of a promoter to the DNA.

47. The non-human mammal according to claim 46, wherein the mammal is a rodent.

48. The non-human mammal according to claim 47, wherein the rodent is a mouse.

49. A method for screening a compound or a salt thereof for promoting or inhibiting the activity of the promoter to the DNA according to claim 7 or 10, which comprises administering test compound to the non-human mammal according to claim 46 and detecting the expression of the reporter gene.


SEQUENCE LISTING (25 sequences)

SEQ ID NO: 1 - DNA, 513 bp, Human
cctggacggagcccctacctctgcaaagatgacttggagacaggccgtcctgctgtcttgcttctccgccgtggtgctcctgtctatgctgagagagggaaccagtgtatctgtgggcaccatgcagatggcgggagaagaggcgagtgaagatgcaaaacagaagattttcatgcaggaatcagatgcctcgaatttcctcaagaggcgcggcaagcggtcccccaagtcccgagatgaggtcaatgtggaaaacaggcagaagcttcgggttgatgagctgcggagagaatattacgaggaacaaaggaatgaatttgagaacttcgtggaggaacaaaacgatgagcaggaagagaggagccgggaggctgtggagcagtggcgccagtggcactatgacggcctgcacccatcctatctctacaaccgccaccacacctgatcccatcctgaagccggccaagaagacaaagcttgtagcaccattggcatccccgtgttccagcaatc

SEQ ID NO: 2 - protein, 138 aa, Human
MTWRQAVLLSCFSAVVLLSMLREGTSVSVGTMQMAGEEASEDAKQKIFMQESDASNFLKRRGKRSPKSRDEVNVENRQKLRVDELRREYYEEQRNEFENFVEEQNDEQEERSREAVEQWRQWHYDGLHPSYLYNRHHT

SEQ ID NO: 3 - DNA, 414 bp, Human
atgacttggagacaggccgtcctgctgtcttgcttctccgccgtggtgctcctgtctatgctgagagagggaaccagtgtatctgtgggcaccatgcagatggcgggagaagaggcgagtgaagatgcaaaacagaagattttcatgcaggaatcagatgcctcgaatttcctcaagaggcgcggcaagcggtcccccaagtcccgagatgaggtcaatgtggaaaacaggcagaagcttcgggttgatgagctgcggagagaatattacgaggaacaaaggaatgaatttgagaacttcgtggaggaacaaaacgatgagcaggaagagaggagccgggaggctgtggagcagtggcgccagtggcactatgacggcctgcacccatcctatctctacaaccgccaccacacc

SEQ ID NO: 4 - protein, 110 aa, Human
VGTMQMAGEEASEDAKQKIFMQESDASNFLKRRGKRSPKSRDEVNVENRQKLRVDELRREYYEEQRNEFENFVEEQNDEQEERSREAVEQWRQWHYDGLHPSYLYNRHHT

SEQ ID NO: 5 - DNA, 330 bp, Human
gtgggcaccatgcagatggcgggagaagaggcgagtgaagatgcaaaacagaagattttcatgcaggaatcagatgcctcgaatttcctcaagaggcgcggcaagcggtcccccaagtcccgagatgaggtcaatgtggaaaacaggcagaagcttcgggttgatgagctgcggagagaatattacgaggaacaaaggaatgaatttgagaacttcgtggaggaacaaaacgatgagcaggaagagaggagccgggaggctgtggagcagtggcgccagtggcactatgacggcctgcacccatcctatctctacaaccgccaccacacc

SEQ ID NO: 6 - protein, 74 aa, Human
SPKSRDEVNVENRQKLRVDELRREYYEEQRNEFENFVEEQNDEQEERSREAVEQWRQWHYDGLHPSYLYNRHHT

SEQ ID NO: 7 - DNA, 222 bp, Human
tcccccaagtcccgagatgaggtcaatgtggaaaacaggcagaagcttcgggttgatgagctgcggagagaatattacgaggaacaaaggaatgaatttgagaacttcgtggaggaacaaaacgatgagcaggaagagaggagccgggaggctgtggagcagtggcgccagtggcactatgacggcctgcacccatcctatctctacaaccgccaccacacc

SEQ ID NO: 8 - protein, 33 aa, Human
VGTMQMAGEEASEDAKQKIFMQESDASNFLKRR

SEQ ID NO: 9 - DNA, 99 bp, Human
gtgggcaccatgcagatggcgggagaagaggcgagtgaagatgcaaaacagaagattttcatgcaggaatcagatgcctcgaatttcctcaagaggcgc

SEQ ID NO: 10 - DNA, 20 bp, Artificial  Sequence
  primer
cctggacggagcccctacct

SEQ ID NO: 11 - DNA, 22 bp, Artificial  Sequence
  primer
gattgctggaacacggggatgc

SEQ ID NO: 12 - DNA, 23 bp, Artificial  Sequence
  primer
aggcagaagcttcgggttgatga

SEQ ID NO: 13 - DNA, 22 bp, Artificial  Sequence
  primer
aggggaagccactgtaggtttg

SEQ ID NO: 14 - DNA, 40 bp, Artificial  Sequence
  primer
ctacgaattccaccatgacttggagacaggccgtcctgct

SEQ ID NO: 15 - DNA, 36 bp, Artificial  Sequence
  primer
ttcaggtcgacggtgtggtggcggttgtagagatag

SEQ ID NO: 16 - protein, 147 aa, Artificial  Sequence
  Human DRL138h-FLAG fusion protein
MTWRQAVLLSCFSAVVLLSMLREGTSVSVGTMQMAGEEASEDAKQKIFMQESDASNFLKRRGKRSPKSRDEVNVENRQKLRVDELRREYYEEQRNEFENFVEEQNDEQEERSREAVEQWRQWHYDGLHPSYLYNRHHTVDYKDDDDK

SEQ ID NO: 17 - DNA, 441 bp, Artificial  Sequence
  DNA fragment encoding Human DRL138h-FLAG fusion protein
atgacttggagacaggccgtcctgctgtcttgcttctccgccgtggtgctcctgtctatgctgagagagggaaccagtgtatctgtgggcaccatgcagatggcgggagaagaggcgagtgaagatgcaaaacagaagattttcatgcaggaatcagatgcctcgaatttcctcaagaggcgcggcaagcggtcccccaagtcccgagatgaggtcaatgtggaaaacaggcagaagcttcgggttgatgagctgcggagagaatattacgaggaacaaaggaatgaatttgagaacttcgtggaggaacaaaacgatgagcaggaagagaggagccgggaggctgtggagcagtggcgccagtggcactatgacggcctgcacccatcctatctctacaaccgccaccacaccgtcgactacaaggacgacgatgacaag

SEQ ID NO: 18 - protein, 138 aa, Mouse
MSWRRVILLSSLLALVLLCMLQEGTSASVGSRQAAAEGVQEGVKQKIFMQESDASNFLKRRGKRSPKSRDEVNAENRQRLRDDELRREYYEEQRNEFENFVEEQRDEQEERTREAVEQWRQWHYDGLYPSYLYNRQNI

SEQ ID NO: 19 - DNA, 414 bp, Human
atgtcctggagacgggtcattctcctgtcatctctcttggccctggtgctcctgtgtatgctacaggaggggaccagcgcttctgtggggagcaggcaggcagctgcagagggggtgcaggaaggtgtgaaacagaagattttcatgcaagaatctgatgcctccaatttcctcaagaggcgtggcaagcggtctcctaagtcccgagatgaagttaatgcggaaaacagacagaggctgcgggatgatgagctgcggagggagtattacgaggagcaaaggaacgagtttgagaacttcgtggaggaacagagagatgagcaggaagagaggacccgggaggctgtggagcagtggcgccagtggcattatgatggcctgtatccttcctacctctacaaccgccaaaacatc

SEQ ID NO: 20 - protein, 110 aa, Mouse
VGSRQAAAEGVQEGVKQKIFMQESDASNFLKRRGKRSPKSRDEVNAENRQRLRDDELRREYYEEQRNEFENFVEEQRDEQEERTREAVEQWRQWHYDGLYPSYLYNRQNI

SEQ ID NO: 21 - DNA, 330 bp, Human
gtggggagcaggcaggcagctgcagagggggtgcaggaaggtgtgaaacagaagattttcatgcaagaatctgatgcctccaatttcctcaagaggcgtggcaagcggtctcctaagtcccgagatgaagttaatgcggaaaacagacagaggctgcgggatgatgagctgcggagggagtattacgaggagcaaaggaacgagtttgagaacttcgtggaggaacagagagatgagcaggaagagaggacccgggaggctgtggagcagtggcgccagtggcattatgatggcctgtatccttcctacctctacaaccgccaaaacatc

SEQ ID NO: 22 - protein, 74 aa, Mouse
SPKSRDEVNAENRQRLRDDELRREYYEEQRNEFENFVEEQRDEQEERTREAVEQWRQWHYDGLYPSYLYNRQNI

SEQ ID NO: 23 - DNA, 222 bp, Human
tctcctaagtcccgagatgaagttaatgcggaaaacagacagaggctgcgggatgatgagctgcggagggagtattacgaggagcaaaggaacgagtttgagaacttcgtggaggaacagagagatgagcaggaagagaggacccgggaggctgtggagcagtggcgccagtggcattatgatggcctgtatccttcctacctctacaaccgccaaaacatc

SEQ ID NO: 24 - protein, 33 aa, Mouse
VGSRQAAAEGVQEGVKQKIFMQESDASNFLKRR

SEQ ID NO: 25 - DNA, 99 bp, Human
gtggggagcaggcaggcagctgcagagggggtgcaggaaggtgtgaaacagaagattttcatgcaagaatctgatgcctccaatttcctcaagaggcgt